(19) United States
(12) Patent Application Publication
(10) Pub. No.: US 2022/0002674 A1
(43) Pub. Date: Jan. 6, 2022

(54) T CELLS WITH SUICIDE SWITCH

(71) Applicant: Bellicum Pharmaceuticals, Inc., Houston, TX (US)

(72) Inventors: Xiaoou Linnette Zhou, Houston, TX (US); Joanne Louise Shaw, Houston, TX (US); Aaron Edward Foster, Houston, TX (US); Madhavi Anumula, Sugar Land, TX (US); Matthew R. Collinson-Pautz, Houston, TX (US)

(21) Appl. No.: 17/288,845

(22) PCT Filed: Oct. 29, 2019
(86) PCT No.: PCT/US19/58664

§ 371 Date: Apr. 26, 2021

Related U.S. Application Data
(60) Provisional application No. 62/753,688, filed on Oct. 31, 2018.

(51) Int. Cl. C12N 5/0783; C12N 9/64; C12N 9/90; A61K 35/17

(52) U.S. Cl. CPC C12N 5/0638; C12Y 304/22062; C12N 9/6472; C12N 2501/998; A61K 35/17; C12N 2510/00; C12N 2501/2302; C12N 9/90

(57) ABSTRACT

We disclose various improvements for compositions of genetically-modified T cells which include a suicide switch. For instance, the composition may comprise CD4+ T cells and CD8+ T cells, wherein the ratio of CD4+ T cells to CD8+ T cells is less than 2.

### REFERENCE TO RELATED APPLICATION

This application claims the benefit of the filing date of U.S. Provisional Application No. 62/753,688 filed, Oct. 31, 2018, the contents of which are incorporated by reference in their entirety for all purposes.

### TECHNICAL FIELD

The invention is in the field of cell transplantation, and techniques useful for eliminating transplanted T cells in a recipient e.g. if Graft versus host disease (GVHD) develops.

### BACKGROUND

During HSCT allogeneic haematopoietic stem cells (HSCs) are derived from a healthy donor and transplanted to a patient. This treatment can be potentially curative for malignant and non-malignant conditions. Unfortunately, however, HSCT can be associated with prolonged post-transplantation immunodeficiency, especially after extensive treatment for underlying malignancies and the use of T-cell-depleted grafts. Considerable time (about 1-2 years) is needed for the complete regeneration of the T cell and B cell compartments, especially when the thymus has lost most of its function owing to age or prior therapies. GVHD and the immunosuppressive drugs used for its prevention can also severely delay immune reconstitution. In addition, relapse remains the most common cause of failure of matched related or unrelated allogeneic HCT.

To treat and prevent relapse, donor lymphocyte administration or infusion is often used after HSCT or HCT. However, with the exception of chronic myeloid leukemia (CML), the efficacy of donor lymphocyte infusions is poor in hematologic malignancies, especially when the relapses occur within the first 12 months of transplant. In addition to providing immune reconstitution and protection to patients, alloreactive T cells can provide potent anti-cancer treatment effects; however, these T cells pose a serious risk of GVHD in the recipient.

The occurrence of GVHD can limit the number of infusions and the number of cells infused, or can warrant an immunosuppressive therapy. This may lead to a high rate of post-transplant infectious complications and a high incidence of disease relapse. Further, it reduces the efficacy of therapeutic cells to treat cancer relapse. Hence, there is a need for better strategies to harness the immunity and anti-tumor potential of the therapeutic cells without the concomitant GVHD and other complications.

One such strategy is the use of genetically modified donor T cells containing a suicide gene switch which can be used to eliminate the cells if complications such as GVHD arise. One such therapy is known as ‘BPX-501’ or ‘rivogenlecleucel’. BPX-501 is based on donor T cells modified to contain inducible caspase 9 (the ‘CaspaCIDe®’ safety switch) which triggers apoptosis of alloreactive T cells in vivo upon exposure to rimiducid (formerly known as AP1903).

There is a need, however, to provide therapeutic cells that have low GVHD rates such that the need to use the suicide switch is reduced. In addition, it will also be beneficial to have mechanisms in which reactive GVHD-causing cells are eliminated while non-reactive cells are not. Such improvements will increase the activity and therapeutic potential of the cells.

### SUMMARY

We provide genetically-modified T cells which have two advantageous properties. Firstly, they have been shown to give rise to GVHD less frequently than T cells used previously (including genetically-modified T cells). Secondly, they are easily and quickly eliminated in vivo by administering a trigger for the suicide switch.

We provide compositions comprising genetically-modified T cells, wherein: (i) the genetically-modified T cells express a suicide switch; and (ii) about 25% to 60% of the genetically-modified T cells are naïve T cells. In certain embodiments, about 30% to 60% of the genetically-modified T cells are naïve T cells. In certain embodiments, about 30% to 60% of the genetically-modified T cells are naïve T cell. Ideally, the composition comprises CD4+ T cells and CD8+ T cells, wherein the ratio of CD4+ T cells to CD8+ T cells is less than 2, or less than 1, or less than 0.5. In certain embodiments, at least 10% of the genetically-modified CD8+ T cells are terminal effector memory T cells and/or no more than 58% of the genetically modified T cells are naïve T cells, or where at least 30% of the genetically-modified CD8+ T cells are terminal effector memory T cells and/or no more than 50% of the genetically modified T cells are naïve T cells. In certain embodiments, the genetically-modified T cells display a range of expression levels of the cell surface transgene marker, wherein the range is at least 10-fold, wherein the expression levels are measured by flow cytometry, and wherein the expression levels are measured as mean fluorescence intensity (MFI) values. In certain embodiments, the range is at least 100-fold. In certain embodiments, the genetically-modified T cells display a range of sensitivities to a trigger molecule, such that exposure of the cells to a particular concentration of the trigger molecule leads to death of at least 10% of the cells but permits at least 10% of the cells to survive. The suicide switch can comprise caspase-9. In certain embodiments, the suicide switch comprises a FKBP12 region, a FKBP12 variant region, a FKBP12-Rapamycin Binding (FRB) or FRB variant region. In certain embodiments, the trigger molecule is rapamycin, a rapalog, AP1903, AP20187, or AP1510. In certain embodiments, the FKBP12 variant region has an amino acid substitution at position 36 selected from the group consisting of valine, leucine, isoleuceine and alanine. In certain embodiments, the FKBP12 variant region comprises two copies of FKBP12v36. The genetically modified T-cells can be human cells. In certain embodiments, (i) the genetically-modified T cells express the iCasp9 suicide switch linked to the ΔCD19 marker, inserted into the genome of the T cells by retroviral transduction; (ii) the genetically-modified T cells have at least a 10-fold range of expression levels of a ΔCD19 cell surface marker measured by flow cytometry; (iii) the composition includes a greater number of CD8+ the genetically-modified T cells than CD4+ the genetically-modified T cells; (iv) the composition includes genetically-modified terminal effector memory T cells, genetically-modified T effector memory cells, and genetically-modified T central memory cells; (v) less than 50% of the genetically-modified T cells are naïve T cells; and (vi) the T cells were obtained from a human donor and were not subjected to a step of allodepletion. In certain embodiments, the genetically-modified T cells have an average vector copy number (VCN) of about 1 to 10 per cell. In certain embodiments, the genetically-modified T cells have an average vector copy number (VCN) of about 1 to 7 per cell. In certain embodiments, the genetically-modified T cells have an average vector copy number (VCN) of about 2 to 6 per cell.

Also provided herein is a composition comprising genetically modified CD3+ T cells, wherein the genetically modified CD3+ T cells comprise about 20% to about 40% CD4+ T cells and about 60% to about 80% CD8+ T cells, wherein (i) the modified CD4+ T cells comprise (a) about 25% to about 45% naïve cells, (b) about 15% to about 30% T-central memory (CM) cells, (c) about 15% to about 30% T-effector memory (EM) cells, (d) about 2% to about 15% terminal effector memory (TEMRA) cells; and (ii) the modified CD8+ T cells comprise (a) about 20% to about 60% naïve cells, (b) about 1% to about 10% CM cells, (c) about 1% to about 15% EM cells, and (d) about 10% to about 15% TEMRA cells.

Also provided herein is a composition comprising genetically modified CD3+ T cells, wherein (i) about 20% to 40% of the T cells in the composition are CD8+ naïve cells; (ii) about 1% to 20% of the T cells in the composition are CD8+ CM cells; (iii) about 1% to 20% of the T cells in the composition are CD8+ EM cells; and (iv) about 5% to 40% of the T cells in the composition are CD8+ TEMRA cells. In certain embodiments, about 20% to 30% of the T cells in the composition are CD8+naïve cells. In certain embodiments, about 1% to 10% of the T cells in the composition are CD8+CM cells. In certain embodiments, about 1% to 10% of the T cells in the composition are CD8+ EM cells. In certain embodiments, about 10% to 30% of the T cells in the composition are CD8+ TEMRA cells.

Also provided herein is a composition comprising genetically modified CD3+ T cells, wherein (i) about 5% to 20% of the T cells in the composition are CD4+ naïve cells; (ii) about 1% to 10% of the T cells in the composition are CD4+ CM cells; (iii) about 1% to 10% of the T cells in the composition are CD4+ EM cells; and (iv) about 1% to 5% of the T cells in the composition are CD4+ TEMRA cells. In certain embodiments, 5% to 15% of the T cells are CD4+ naïve cells. In certain embodiments, 1% to 7% of the T cells are CD4+ CM cells. In certain embodiments, 1% to 10% of the T cells are CD4+ EM cells.

Also provided herein is a method of treating a subject comprising a step of introducing into the subject a composition of any of the above claims.

Also provided herein is a method for treating a subject, comprising a step of administering to the subject a pharmacological agent, wherein: (i) the subject has previously received an infusion of genetically-modified T cells according to any of the above compositions; (ii) the pharmacological agent triggers the suicide switch; and (iii) the pharmacological agent is delivered at a dose which is high enough to kill at least 10% of genetically-modified T cells present in the subject, but low enough that at least 10% of genetically-modified T cells present in the subject survive.

Also provided herein is a process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of terminal effector memory T cells, central memory T cells, and/or effector memory T cells relative to naïve T cells. In certain embodiments, the method favours enrichment of terminal effector memory T cells relative to naïve T cells.

Also provided herein is a process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of CD8+ T cells relative to CD4+ T cells.

Also provided herein is a process for preparing genetically-modified T cells, comprising steps of: (i) culturing donor T cells in the presence of activating concentrations of IL 2, anti-CD3 antibody, and anti-CD28 antibody; (ii) after allowing a period of culture, adding further IL 2; (iii) after allowing a period of culture, introducing into the T cells DNA encoding both a suicide switch and a selectable marker; (iv) after allowing a period of culture, adding further IL 2; (v) selecting cells which express the selectable marker; (vi) after allowing a period of culture, adding further IL 2; (vii) harvesting the genetically-modified T cells; provided that steps (iv) and (vi) are optional. In certain embodiments the genetically-modified T cells have an average-VCN of about 1 to 10 per cell. In certain embodiments, the genetically-modified T cells have an average VCN of about 1 to 7 per cell. In certain embodiments, step (i) occurs at the start of the process (day 1). In certain embodiments, step (iv) occurs on day 3 of the process. In certain embodiments, step (vi) occurs on day 6 of the process. In certain embodiments, at the end of the process, at least 50% of the cells are transduced and viable. In certain embodiments, at the end of the process, at least 90% of the cells are transduced and viable

We also provide compositions comprising genetically-modified CD8+ T cells, wherein: (i) the genetically-modified CD8+ T cells express a suicide switch; and (ii) at least 1% to about 30% of the genetically-modified CD8+ T cells are terminal effector memory T cells and/or no more than 58% of the genetically-modified CD8+ T cells are naïve T cells.

We also provide compositions comprising genetically-modified T cells, wherein: (i) the genetically-modified T cells express a suicide switch and a cell surface transgene marker; and (ii) the genetically-modified T cells display a range of expression levels of the cell surface transgene marker, wherein the range is at least 10-fold. The expression levels are measured as MFI values by flow cytometry. The ratio of CD4+ T cells to CD8+ T cells in this composition is preferably less than 2.

We also provide compositions comprising genetically-modified T cells, wherein: (i) the genetically-modified T cells express a suicide switch which can lead to cell death when the cells are exposed to a trigger molecule; and (ii) the genetically-modified T cells display a range of sensitivities to the trigger molecule, such that exposure of the cells to a particular concentration of the trigger molecule leads to death of at least 10% of the cells but permits at least 10% of the cells to survive.

In some embodiments, the genetically-modified T cells have an average vector copy number (VCN) of about 1 to 10 per cell, or about 1 to 8 per cell, or about 1 to 7 per cell, or about 1 to 5.

In some embodiments, the suicide switch comprises a FKBP12 region, a FKBP12 variant region, a FKBP12-Rapamycin Binding (FRB) or FRB variant region. In some embodiments, the FKBP12 variant region has an amino acid substitution at position 36 selected from the group consisting of valine, leucine, isoleuceine and alanine. In some embodiments, the trigger molecule is rapamycin, a rapalog, AP1903, AP20187, or AP1510.

We also provide a process for preparing genetically-modified T cells, comprising steps of: (i) culturing donor T cells in the presence of activating concentrations of IL-2, anti-CD3 antibody, and anti-CD28 antibody; (ii) after allowing a period of culture, adding further IL-2; (iii) after allowing a period of culture, introducing into the T cells DNA encoding both a suicide switch and a selectable marker; (iv) after allowing a period of culture, adding further IL-2; (v) selecting cells which express the selectable marker; (vi) after allowing a period of culture, adding further IL-2; (vii) harvesting the genetically-modified T cells; provided that steps (iv) and (vi) are optional (but preferably one is, or more preferably both are, performed). The harvested T cells can be used as disclosed herein.

We also provide a process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of terminal effector memory T cells, central memory T cells, and/or effector memory T cells relative to naïve T cells. Preferably the method favours enrichment of terminal effector memory T cells relative to naïve T cells. Steps (i) and (ii) need not be performed in that order, and step (i) can occur during step (ii).

We also provide a process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of CD8+ T cells relative to CD4+ T cells. Steps (i) and (ii) need not be performed in that order, and step (i) can occur during step (ii).

We also provide a method for treating a subject, comprising a step of introducing into the subject a composition of genetically-modified T cells as described above. Similarly, we provide (a) a composition of genetically-modified T cells as described above, for use in treating a subject and (b) the use of a composition of genetically-modified T cells as described above in the manufacture of a medicament for treating a subject. The subject is ideally a human subject, and will usually receive the cells in conjunction with HCT or HSCT.

We also provide a method for treating a subject, comprising a step of administering to the subject a pharmacological agent, wherein: (i) the subject has previously received an infusion of genetically-modified T cells as described herein; (ii) the pharmacological agent triggers the suicide switch; and (iii) the pharmacological agent is delivered at a dose which is high enough to kill at least 10% of genetically-modified T cells present in the subject, but low enough that at least 10% of genetically-modified T cells present in the subject survive.

### BRIEF DESCRIPTION OF DRAWINGS

FIGS. 1A & 1B show CD19 surface expression on non-transduced T cells (NT-T), AOS-1 and sorted cells based on their CD19 levels. Cells were gated on 7AAD−/AnnV−/CD3+ population/CD19-APC. Each shape indicates cells from the same individual donor.

FIGS. 2A & 2B show the proportion of CD8 and CD4 subsets among non-transduced cells, AOS-1 and sorted cells based on their CD19 levels. Each shape indicates cells from the same individual donor.

FIG. 3 shows the composition of memory and effector cells among non-transduced cells, AOS-1 and CD19+ sorted cells in CD3+ cells. Each bar represents in order, from top to bottom, TEMRA, EM, CM, and Naïve cells.

FIG. 4 shows the composition of memory and effector cells among non-transduced cells, AOS-1 and CD19′ sorted cells in CD4+ and CD8+ subsets. Each bar represents in order, from top to bottom, TEMRA, EM, CM, and Naïve cells.

FIGS. 5A & 5B provide results of apoptosis induced by rimiducid in a 4-hour apoptosis assay with 10 nM rimiducid. Cells were stained with 7AAD/AnnV/CD3/CD19. Each shape indicates cells from the same individual donor.

FIG. 6 depicts the correlation of rimiducid-induced apoptosis (measured as MFI of CD19+ of viable CD3+ cells) with the intensity of the iC9-ΔCD19 transgene expression.

FIG. 7 depicts the correlation of rimiducid-induced apoptosis (measured as % of AnnV+ cells) with the intensity of the iC9-ΔCD19 transgene expression.

FIG. 8 provides a western blot showing that surface expression of CD19 is related to caspase-9 protein level.

FIG. 9 shows the correlation of rimiducid-induced apoptosis (measured as killing efficacy) with the intensity of the iC9-ΔCD19 transgene expression.

FIG. 10 depicts a graph showing that iC9-expressing cells with higher CD19 MFI carry higher vector copy number.

FIG. 11 depicts graphs showing the up-regulation of CD25, CD69, and PD-1 on T cells upon ex vivo stimulation.

FIG. 12 depicts graphs showing show up-regulation of CD25, CD69, and PD-1 on CD4+ and CD8⁺ T cell subsets upon ex vivo stimulation.

FIGS. 13A, 13B & 13C show results that demonstrate that rimiducid-induced apoptosis can be enhanced when cells are activated. Cells were re-activated with anti-CD3/anti-CD28 and apoptosis was induced in 4-hours apoptosis assay with 10 nM rimiducid.

FIG. 14A provides a western blot showing that killing efficacy is related to caspase-9 and anti-apoptotic protein level. FIG. 14B depicts graphs showing the transition of naïve and memory populations upon activation. In FIG. 14B, each bar represents in order, from top to bottom, TEMRA, EM, CM, and Naïve cells.

FIG. 15 provides dorsal IVIS imaging of iC9-T cells.

FIG. 16 provides ventral IVIS imaging of iC9-T cells.

FIG. 17 depicts IVIS imaging of T cells showing rimiducid dependent killing.

FIG. 18 shows the detection of iC9-T cells in spleen and the selective elimination of the T cells with higher expression and transduction of iC9.

FIGS. 19A & 19B show the peak expansion of AOS-1 cells in CD3+ T cells and the composition of memory and effector cells among AOS-1 cells. In FIG. 19B (right), each bar represents in order, from top to bottom, TEMRA, EM, CM, and Naïve cells.

FIG. 20 depicts the reconstitution and the composition of memory and effector cells among of endogenous T cells. In FIG. 20 (right), each bar represents in order, from top to bottom, TEMRA, EM, CM, and Naïve cells.

FIG. 21 depicts engraftment and the composition of memory & effector cells among AOS-1 cells. In FIG. 21 (right), each bar represents in order, from top to bottom, TEMRA, EM, CM, and Naïve cells.

FIG. 22 provides the reconstitution of B cells and NK cells.

FIG. 23 depicts the anti-tumor activity from infused AOS-1 products.

FIG. 24 shows the detection of tumor associated antigens post infusion.

FIG. 25 shows that AOS-1 demonstrates higher TCR skewing compared to endogenous T cells.

FIG. 26 depicts skewing of immune repertoire in endogenous and AOS-1 cells. For each clone, the bars represent, in order from left to right, Day 80, Day 128, and Day 180.

FIG. 27 shows the effect of 5 mg/kg rimiducid administered on day 0 to mice who received 1.5×10⁷ AOS-1 cells on day −1. Figures are means from 40 mice. FIG. 27A shows absolute cell numbers, and FIG. 27B shows numbers relative to the time of rimiducid administration.

FIG. 28 shows VCN Distribution for AOS-1 cells

FIG. 29 shows AOS-1 cells IFN-γ production after anti-CD3 stimulation

### DETAILED DESCRIPTION

T cells from a donor can be genetically-modified prior to their administration to a recipient (e.g. as part of HCT or HSCT) to provide them with a suicide switch. In the event that the T cells cause complications in the recipient (e.g. GVHD) the suicide switch can be triggered, leading to eradication of the genetically-modified cells.

T cells

T cells which are genetically modified as disclosed herein are useful for administering to subjects who can benefit from donor lymphocyte administration. These subjects will typically be humans, so the invention will typically be performed using human T cells.

The T cells can be derived from any healthy donor. The donor will generally be an adult (at least 18 years old) but children are also suitable as T cell donors (e.g. see Styczynski 2018, Transfus Apher Sci 57(3):323-330).

A suitable process for obtaining T cells from a donor is described in the published protocol which accompanied Di Stasi et al. (2011) N Engl J Med 365:1673-83 (‘The Protocol’). In general terms, T cells are obtained from a donor, subjected to genetic modification and selection, and can then be administered to recipient subjects. A useful source of T cells is the donor's peripheral blood. Peripheral blood samples will generally be subjected to leukapheresis to provide a sample enriched for white blood cells. This enriched sample (also known as a leukopak) can be composed of a variety of blood cells including monocytes, lymphocytes, platelets, plasma, and red cells. A leukopak typically contains a higher concentration of cells as compared to venipuncture or buffy coat products.

Although the sample may be subjected to allodepletion (as discussed in The Protocol), it is preferred that the sample is not subjected to allodepletion. Preferred samples are thus alloreplete, as discussed in Zhou et al. (2015) Blood 125:4103-13. These populations provide a more robust T cell repertoire for providing the therapeutic advantages of the donor cells. Preferred compositions of the invention are thus not T cell allodepleted, and have not been subject to a step of allodepletion.

Donor T cells are generally cultured (usually under activating conditions e.g. using anti-CD3 and/or anti-CD28 antibodies, optionally with IL-2) prior to being genetically modified. This step provides higher yields of T cells at the end of the modification process.

The T cells can be transduced using a viral vector encoding the suicide switch of interest (see below). Suitable transduction techniques are disclosed in The Protocol and may involve fibronectin fragment CH-296. As an alternative to transduction using a viral vector, cells can be transfected with any suitable method known in the art such as with DNA encoding the suicide switch of interest and a cell surface transgene marker of interest e.g. using calcium phosphate, cationic polymers (such as PEI), magnetic beads, electroporation and commercial lipid-based reagents such as Lipofectamine™ and Fugene™. One result of the transduction/transfection step is that various donor T cells will now be genetically-modified T cells which can express the suicide switch of interest.

A preferred viral vector for transduction is the retroviral vector disclosed by Tey et al. (2007) Biol Blood Marrow Transpl 13:913-24 and by Di Stasi et al. (2011) supra. This vector is based on Gibbon ape leukemia virus (Gal-V) pseudotyped retrovirus encoding an iCasp9 suicide switch and a ΔCD19 cell surface transgene marker (see further below). It can be produced in the PG13 packaging cell line, as discussed by Tey et al. (2007) supra. Other viral vectors encoding the desired proteins can also be used. Retroviral vectors are preferred, particularly those which can provide a high copy number of proviral integrants per cell.

After transduction/transfection, cells can be separated from transduction/transfection materials and cultured again, to permit the genetically-modified T cells to expand. T cells can be expanded so that a desired minimum number of genetically-modified T cells is achieved e.g. as disclosed in The Protocol.

Genetically-modified T cells can then be selected from the population of cells which has been obtained. The suicide switch will usually not be suitable for positive selection of desired T cells, so it is preferred that the genetically-modified T cells should also express a cell surface transgene marker of interest (see below). Cells which express this surface marker can be selected e.g. using immunomagnetic techniques. For instance, paramagnetic beads conjugated to monoclonal antibodies which recognise the cell surface transgene marker of interest can be used, as disclosed in The Protocol e.g. using a CliniMACS system (available from Miltenyi Biotec).

In an alternative procedure, genetically-modified T cells are selected after a step of transduction, are cultured, and are then fed. Thus the order of transduction, feeding, and selection can be varied.

The result of these procedures is a composition containing donor T cells which have been genetically modified and which can thus express the suicide switch of interest (and, typically, the cell surface transgene marker of interest). These genetically-modified T cells can be administered to a recipient, but they will usually be cryopreserved (optionally after further expansion) before being administered.

### CD4+ and CD8+ Cells

The composition can include CD4+ and CD8+ T cells, and ideally the population of genetically-modified CD3+ T cells within the composition includes CD4+ cells and CD8+ cells. Whereas the ratio of CD4+ cells to CD8+ cells in a leukopak is typically above 2, in some embodiments the ratio of genetically-modified CD4+ cells to genetically-modified CD8+ cells in a composition of the invention is less than 2 e.g. less than 1.5. Ideally there are more genetically-modified CD8+ T cells than genetically-modified CD4+ T cells in the composition i.e. the ratio of CD4+ cells to CD8+ cells is less than 1 e.g. less than 0.9, less than 0.8, less than 0.7, less than 0.6, or preferably even less than 0.5. Thus the overall procedure starting from donor cells and producing genetically-modified T cells ideally enriches for CD8+ cells T cells relative to CD4+ T cells. Preferably at least 60% of the genetically-modified T cells are CD8+ T cells, and more preferably at least 65%. Within the population of genetically-modified CD3+ T cells a preferred range for CD8+ T cells is between 55-75% e.g. from 63-73%. The proportions of CD8+ and CD4+ T cells can easily be assessed by flow cytometry. Methods for sorting and counting CD4+ and CD8+ T cells are conventional in the art.

### Memory T Cell Subsets (See Mahnke et al. (2013) Eur J Immunol 43:2797-809)

The population of genetically-modified CD3+ T cells within the composition can include terminal effector memory T cells (defined as CD45RA+CD45RO−CCR7− cells; ‘TEMRA’), T-effector memory cells (defined as CD45RA-CD45RO+CCR7− cells; ‘EM’), T-central memory cells (defined as CD45RA-CD45RO+CCR7+ cells; ‘CM’), and naïve T cells (defined as CD45RA+CD45RO−CCR7+ cells). These cells can be assessed by flow cytometry using the CD45RA/RO and CCR7 markers. Labelled reagents which recognise CCR7 and which can distinguish between the CD45RA and CD45RO isoforms are readily available from commercial suppliers.

An average leukopak typically contains ˜20% each of terminal effector and T-effector memory cells. The overall procedure from donor cells to genetically-modified T cells may enrich for terminal effector memory T cells relative to T-effector memory cells.

In some embodiments, less than 60% of the genetically-modified T cells are naïve T cells e.g. less than 58%, preferably less than 55%, and more preferably less than 50%. Within the population of genetically-modified CD3+ T cells a preferred range for naïve T cells is between 30-60%, more preferably 42-49%, and most preferably from 43-46%. This proportion of naïve T cells has been seen to correlate with favourable outcomes in T cell recipients. Naïve EM cells can be assessed by flow cytometry using the CD45RA/RO and CCR7 markers.

In some embodiments, at least 23% of the genetically-modified T cells are terminal effector memory (TEMRA) T cells. In such embodiments, the fraction of TEMRA T cells is preferably at least 27%, more preferably at least 30%, and is most preferably at least 33%. In some embodiments a fraction of more than 38% TEMRA T cells has even been observed. Within the population of genetically-modified CD3+ T cells a preferred range for TEMRA T cells is between 23-40% e.g. from 28-40%, or preferably from 33-39% TEMRA cells can be assessed by flow cytometry using the CD45RA/RO and CCR7 markers.

In some embodiments, no more than 17% of the genetically-modified T cells are T-effector memory (EM) cells. In such embodiments, the fraction of EM T cells is preferably less than 16%, more preferably less than 15%, and is most preferably less than 14%. Within the population of genetically-modified CD3+ T cells a preferred range for EM T cells is between 7-17% e.g. from 7-15%, or preferably from 8-13% EM cells can be assessed by flow cytometry using the CD45RA/RO and CCR7 markers.

Within a population of genetically modified T cells, in addition to TEMRA, EM and naïve T cells, the proportion of T-central memory cells is generally <10%.

Looking more particularly at the CD8+ population of genetically-modified T cells, in some embodiments at least 1% to about 30% of the genetically-modified CD8+ T cells are terminal effector memory T cells. In other embodiments, no more than 58% of the genetically-modified CD8+ T cells are naïve T cells. Preferably, there is at least 5% TEMRA and no more than 58% naïve T cells. In. other embodiments, there is at least 5% TEMRA and about at least 25% to no more than 58% naïve T cells. In. other embodiments, there is at least 10% TEMRA and about at least 25% to no more than 58% naïve T cells. In. other embodiments, there is at least 20% TEMRA and about at least 25% to no more than 58% naïve T cells. In. other embodiments, there is at least 5% TEMRA and about at least 30% to no more than 58% naïve T cells. An average leukopak typically contains ˜25% TEMRA CD8+ T cells and ˜60% naïve CD8+ T cells. Thus the overall procedure starting from donor cells and producing genetically-modified T cells ideally enriches memory T cells relative to naïve T cells within the CD8+ fraction.

Where ≥30% of the genetically-modified CD8+ T cells are TEMRA cells, it is preferred that there is at least 35% TEMRA, more preferably at least 40% TEMRA, and most preferably at least 45% TEMRA. In some embodiments a fraction of more than 50% CD8+ TEMRA T cells has even been observed. Within the population of genetically-modified CD3+CD8+ T cells a preferred range for TEMRA T cells is between 30-60% e.g. from 35-55%, or preferably from 40-55%.

Where ≤58% of the genetically-modified CD8+ T cells are naïve T cells, it is preferred that there is at <50% naïve T cells, and more preferably <48% naïve T cells. Within the population of genetically-modified CD3+CD8+ T cells a preferred range for naïve T cells is between 25%-58% e.g. from 30%-55%, or 35-55%, preferably from 38-52%, and most preferably 40-48%.

Within a population of genetically modified CD8+ T cells, in addition to TEMRA, EM and naïve T cells, the proportion of T-central memory cells is generally <4%.

In some embodiments, the population of genetically-modified CD3+ T cells within the composition comprises about 10% to about 40% CD4+ T cells and about 60% to about 90% CD8+ T cells. The population of genetically-modified CD3+ T cells can comprise about 15% to about 40% CD4+ T cells and about 60% to about 85% CD8+ T cells, more preferably about 20% to about 40% CD4+ T cells and about 60% to about 80% CD8+ T cells. In some embodiments, the modified CD4+ T cells comprises about 20% to about 50% naïve cells, about 15% to 40% CM cells, about 15% to 40% EM cells, and about 2% to about 15% TEMRA cells. In some embodiments, the modified CD4+ T cells comprises about 25% to about 45% naïve cells, about 15% to 30% CM cells, about 15% to 30% EM cells, and about 2% to about 15% TEMRA cells. In some embodiments, the modified CD8+ T cells comprises about 20% to about 60% naïve cells, about 1% to 20% CM cells, about 1% to 20% EM cells, and about 10% to about 40% TEMRA cells. In some embodiments, the modified CD8+ T cells comprises about 20% to about 60% naïve cells, about 1% to 10% CM cells, about 1% to 15% EM cells, and about 10% to about 40% TEMRA cells.

In some embodiments the population of genetically-modified CD3+ T cells within the composition comprises about 20% to about 40% CD4+ T cells and about 60% to about 80% CD8+ T cells, wherein the modified CD4+ T cells comprises about 25% to about 45% naïve cells, about 15% to 30% CM cells, about 15% to 30% EM cells, and about 2% to about 15% TEMRA cells; and wherein the modified CD8+ T cells comprises about 20% to about 60% naïve cells, about 1% to 10% CM cells, about 1% to 15% EM cells, and about 10% to about 40% TEMRA cells.

In some embodiments, the invention provides for a composition comprising genetically-modified CD3+ T cells, where about 20% to 40% of the T cells in the composition are CD8+ naïve cells, about 1% to 20% of the T cells in the composition are CD8+ CM cells, about 1% to 20% of the T cells in the composition are CD8 EM cells, and about 5% to 40% of the T cells in the composition are CD8+ TEMRA cells. In some embodiments, the invention provides a composition comprising genetically-modified CD3+ T cells, where about 20% to 30% of the T cells in the composition are CD8+ naïve cells, about 1% to 10% of the T cells in the composition are CD8+ CM cells, about 1% to 10% of the T cells in the composition are CD8+ EM cells, and about 10% to 30% of the T cells in the composition are CD8+ TEMRA cells.

In some embodiments, the invention provides for a composition comprising genetically-modified CD3+ T cells, where about 5% to 20% of the T cells in the composition are CD4+ naïve cells, about 1% to 10% of the T cells in the composition are CD4+ CM cells, about 1% to 10% of the T cells in the composition are CD4+ EM cells, and about 1% to 5% of the T cells in the composition are CD4+ TEMRA cells. In some embodiments, the invention provides a composition comprising genetically-modified CD3+ T cells, where about 5% to 15% of the T cells in the composition are CD4+ naïve cells, about 1% to 7% of the T cells in the composition are CD4+ CM cells, about 1% to 10% of the T cells in the composition are CD4+ EM cells, and about 1% to 5% of the T cells in the composition are CD4+ TEMRA cells.

### Suicide Switches

Genetically-modified T cells of the invention express a suicide switch (also known in the art as an inducible suicide gene or safety switch), which can be used to eradicate the T cells in vivo if desired e.g. if GVHD develops. These switches respond to a trigger, such as a pharmacological agent, which is supplied when it is desired to eradicate the T cells, and which leads to cell death (e.g. by triggering necrosis or apoptosis). These agents can lead to expression of a toxic gene product, but a more rapid response can be obtained if the genetically-modified T cells already express a protein which is switched into a toxic form in response to the agent.

A preferred suicide switch is based on an apoptotic protein which can be triggered by administering a chemical inducer of dimerization to a subject. If the apoptotic protein is fused to a polypeptide sequence which binds to the chemical inducer of dimerization, delivery of this chemical inducer or ligand can bring two apoptotic proteins into proximity such that they trigger apoptosis. For instance, caspase-9 can be fused to a modified human FK-binding protein which can be induced to dimerize in response to the pharmacological agent rimiducid. Delivery of rimiducid to a subject can therefore trigger apoptosis of T cells which express the caspase-9 switch.

Such caspase-9 switches are described in Di Stasi et al. (2011) supra; see also Yagyu et al. (2015) Mol Ther 23(9):1475-85, Rossigloni et al. (2018) Cancer Gene Ther doi.org/10.1038/s41417-018-0034-1, Jones et al. (2014) Front Pharmacol doi.org/10.3389/fphar.2014.00254, U.S. Pat. Nos. 9,932,572, 9,913,882, 9,393,292, and patent application US2015/0328292.

Suicide switches based on Fas or on HSV thymidine kinase are also well known in the art. Suicide switches based on human proteins, such as caspase-9, are preferred because this minimises the risk that cells expressing the switch will be recognised as foreign by a human subject's immune system.

Examples of ligand inducers for the switches include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)

The ligand binding regions incorporated in the safety switches may comprise the FKBP12v36 modified FKBP12 polypeptide, or other suitable FKBP12 variant polypeptides, including variant polypeptides that bind to AP1903, or other synthetic homodimerizers such as, for example, AP20187 or AP2015. Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). AP1903, also known as rimiducid, (CAS Index Name: 2-Piperidinecarboxylic acid, 1-[(2S)-1-oxo-2-(3, 4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]] ester, [2S-[1(R*),2R*[S*[S*[1(R*),2R*]]]]]-(9CI) CAS Registry Number: 195514-63-7; Molecular Formula: C78H98N4O20 Molecular Weight: 1411.65), is a synthetic molecule that has proven safe in healthy volunteers (Iuliucci J D, et al., J Clin Pharmacol. 2001, 41:870-879).

The safety switch may comprise a modified Caspase-9 polypeptide having modified activity, such as, for example, reduced basal activity in the absence of the homodimerizer ligand. Modified Caspase-9 polypeptides are discussed in, for example, U.S. Pat. No. 9,913,882 and US-2015-0328292, supra, and may include, for example, amino acid substitutions at position 330 (e.g., D330E or D330A) or, for example, amino acid substitutions at position 450 (e.g., N405Q), or combinations thereof, including, for example, D330E-N405Q and D330A-N405Q. Caspase-9 polypeptide with lower basal activity have been described previously, e.g. in U.S. Pat. Nos. 9,434,935, 9,932,572 and 9,913,882, and U.S. Patent Application Nos. 62/668,223, 62/756,442, 62/816,799, 15/901,556, 15/888,948. 0090J The most preferred suicide switch for use with the invention is the iCasp9 disclosed by Di Stasi et al. (2011) supra, which consists of the sequence of the human FK506-binding protein (FKBP12) with an F36V mutation, connected through a SGGGS linker to a modified human caspase 9 (CASP9) which lacks its endogenous caspase activation and recruitment domain. The F36V mutation increases the binding affinity of FKBP12 to synthetic homodimerizers AP20187 and AP1903 (rimiducid).

Non-limiting examples of safety switched useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety, including all text, tables and drawings, for all purposes. Multimeric compounds described herein, or pharmaceutically acceptable salts thereof, may be used essentially as discussed in examples provided in these publications, and other examples provided herein.

In various embodiments, the compositions and methods contemplated herein have a transduction efficiency to at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, including any intervening percentages.

In various embodiments, the compositions and methods contemplated herein the average vector copy number (VCN) is at least about 1 to at least about 10.0, at least about 1 to at least about 9, at least about 1 to at least about 8, at least about 1.0 to at least about 7, at least about 1.0 to at least about 5, or at least about 1.0, at least about 1.5, at least about 2.0, at least about 2.5, at least about 3.0, at least about 3.5, at least about 4.0, at least about 4.5, or at least about 5.0.

“Vector copy number” or “VCN” refers to the number of copies of a vector, or portion thereof, in a cell's genome. The average VCN may be determined from a population of cells or from individual cell colonies. Exemplary methods for determining VCN include polymerase chain reaction (PCR) and flow cytometry.

In various embodiments, a population of T cells is transduced with a suicide switch as described herein, wherein at least 50% of T cells in a composition are transduced and viable and wherein the genetically modified T cells have an average vector copy number (VCN) of about 1 to 10 is contemplated.

In some embodiments, a population of T cells is transduced with a suicide switch as described herein, wherein at least 60%, or a least 70%, or at least 80%, or at least 90% of T cells in a composition are transduced and viable and wherein the genetically modified T cells have an average vector copy number (VCN) of about 1 to 10 is contemplated.

In various embodiments, a population of T cells is transduced with a suicide switch as described herein, wherein at least 90% of T cells in a composition are transduced and viable and wherein the genetically modified T cells have an average vector copy number (VCN) of about 1 to 10 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 8 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 7 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 5 is contemplated.

### Cell Surface Transgene Marker

As noted above, expression of the suicide switch will usually not be suitable for positive selection of desired genetically-modified T cells, so it is preferred that these cells should express a cell surface marker of interest. This marker should be expressed from a transgene which is delivered in conjunction with the suicide switch, such that selection of cells based on the cell surface transgene marker also leads to selection of cells which can express the suicide switch.

Ideally, the marker should be a polypeptide which is not expressed by the donor T cells. Moreover, ideally the marker is based on a human proteins as this minimises the risk that cells expressing the marker will be recognised as foreign by a human subject's immune system. For instance, human CD proteins which are not naturally expressed by T cells can be used for this purpose.

The most preferred cell surface transgene marker for use with the invention is the truncated CD19 (ΔCD19) disclosed by Di Stasi et al. (2011) supra, which consists of human CD19 truncated at amino acid 333 to remove most of the intracytoplasmic domain. The extracellular CD19 domain can still be recognised (e.g. in flow cytometry, FACS or MACS) but the potential to trigger intracellular signalling is minimised. CD19 is normally expressed by B cells, rather than by T cells, so selection of CD19+ T cells permits the genetically-modified T cells to be separated from unmodified donor T cells.

We have found that expression levels of the cell surface transgene (and vector copy number after transduction) can vary widely within genetically modified T cells. Moreover, the pharmacokinetic and pharmacodynamics properties of cells differ according to their expression levels of the marker.

The genetically-modified T cells can display at least a 10-fold range of expression levels of the marker i.e. the population includes some cells which display at least 10× less cell surface marker than the cells which express the highest level of the marker. These expression levels can be assessed using flow cytometry because the transgene marker is on the cell surface, e.g. as a mean fluorescence intensity (MFI) value. Thus, the fluorescent signal seen for the marker in a FACS experiment will have a range of at least 10. In practice, ranges much higher than 10 can be achieved e.g. at least 50, at least 100, at least 500, or even 1000 or more.

Expression levels will typically vary continuously across a range of at least 10 (rather than, for instance, being in two groups with expression levels of x and 10×). There is thus a distribution of expression levels, and the population includes some cells whose expression level of the marker is 1/10 of the highest expression level which is observed, with a variety of expression levels distributed within that range (and, typically, also some cells with lower expression levels too). A FACS cell count histogram for expression of the cell surface marker will thus include levels which vary by at least 10-fold, and typically a variety of different expression levels distributed across that range.

By including cells with a range of expression levels of the cell surface transgene marker, there is also a range of expression levels of the suicide switch. We have shown that T cells with high expression levels of the suicide switch are eradicated in vivo more rapidly and more completely when the suicide switch is triggered, while T cells with low expression levels can survive for longer. Moreover, we have found that expression levels of the marker increase when T cells are activated. Without wishing to be bound by theory, by having a range of expression levels in the genetically-modified T cells it seems possible to eliminate the problematic cells (e.g. during GVHD) relatively easily while retaining some genetically-modified T cells which can still provide the benefits of supplying donor T cells. In other words, the range of expression levels permits T cell elimination to be targeted on the activated problematic cells rather than by eliminating all donor T cells regardless of their contribution to GVHD.

Thus genetically-modified T cells within a composition of the invention may display a range of sensitivities to a trigger molecule such as rimiducid. The trigger molecule may thus be used to eradicate only a portion of the T cells (e.g. at least 10%), whereas some of the T cells (e.g. at least 10%) survive. The concentration of the trigger molecule can be selected according to the desired balance of cell death and survival e.g. a higher concentration will be delivered if a higher proportion of T cell eradication is desired. These concentrations can be determined by simple dose-ranging experiments, monitoring levels of cell death in response to the trigger molecule. The concentration which is administered should be high enough to eliminate at least 10% of the target T cells, but not so high that it kills more than 90% of them. The survival rate can be selected to be anywhere within this range of 10-90% survival.

### Co-Expression of Suicide Switch and Cell-Surface Transgene Marker

As noted above, genetically-modified T cells of the invention should express both the suicide switch and the cell surface transgene marker, such that selection of T cells which express the marker also leads to selection of cells which express the suicide switch.

Ideally, the genetically-modified T cells express a fixed ratio of the suicide switch and the cell surface transgene marker. To ensure that the suicide switch and cell-surface transgene marker are expressed together, it is preferred to express them from a single gene, whose translated polypeptide is cleaved to provide both mature polypeptides separately. One way to achieve this is to link the two polypeptide sequences by a 2A-like sequence derived from the Thosea asigna insect virus. This provides for essentially a fixed stoichiometric ratio of expression of the suicide switch and the cell surface marker (a 1:1 ratio if two mature polypeptides are linked by a single 2A-like sequence). By encoding the suicide switch at the 5′ end of the encoding gene, the risk of selecting cells which do not have the suicide switch (e.g. due to premature termination of translation) is minimised. In this manner, expression of the cell surface transgene marker and of the suicide switch run in parallel.

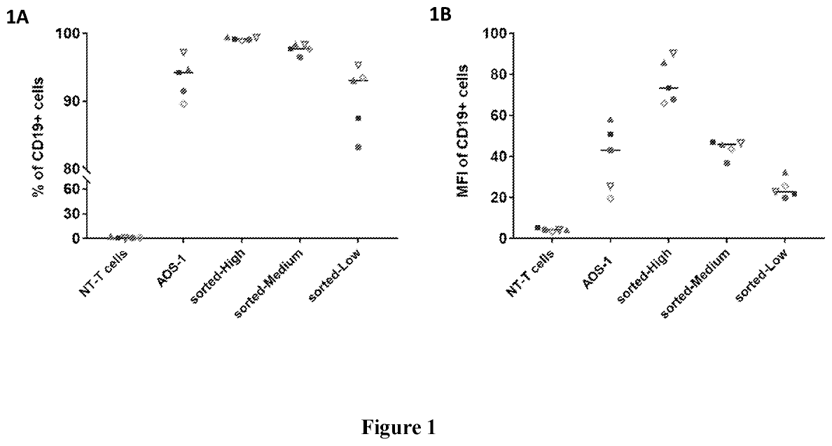

In some embodiments it is envisaged that the suicide switch and the cell surface transgene marker could be the same mature polypeptide, but in practical terms is it simpler that they are separate polypeptides. A preferred gene therefore encodes, from 5′ to 3′: the human FK506-binding protein (FKBP12) with an F36V mutation, connected through a SGGGS linker to a modified human caspase 9 (CASP9) which lacks its endogenous caspase activation and recruitment domain, connected through a 2A-like sequence to the ΔCD19 marker (see FIG. 1C of Di Stasi et al. (2011) supra). Furthermore, and as noted above, it is preferred that this gene is encoded by a retroviral vector as also disclosed by Di Stasi et al. (2011) supra based on Gibbon ape leukemia virus (Gal-V). This system is shown herein to provide an efficient system for selecting genetically-modified T cells and for controlling their apoptosis in humans in vivo.

In various embodiments, the compositions and methods contemplated herein have a transduction efficiency to at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, including any intervening percentages.

In various embodiments, the compositions and methods contemplated herein the average VCN is at least about 1 to at least about 10.0, at least about 1 to at least about 9, at least about 1 to at least about 8, at least about 1.0 to at least about 7, at least about 1.0 to at least about 5, or at least about 1.0, at least about 1.5, at least about 2.0, at least about 2.5, at least about 3.0, at least about 3.5, at least about 4.0, at least about 4.5, or at least about 5.0.

In various embodiments, a population of T cells is transduced with a suicide switch as described herein, wherein at least 50% of T cells in a composition are transduced and viable and wherein the genetically modified T cells have an average vector copy number (VCN) of about 1 to 10 is contemplated.

In some embodiments, a population of T cells is transduced with a suicide switch as described herein, wherein at least 60%, or a least 70%, or at least 80%, or at least 90% of T cells in a composition are transduced and viable and wherein the genetically modified T cells have an average vector copy number (VCN) of about 1 to 10 is contemplated.

In various embodiments, a population of T cells is transduced with a suicide switch as described herein, wherein at least 90% of T cells in a composition are transduced and viable and wherein the genetically modified T cells have an average vector copy number (VCN) of about 1 to 10 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 8 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 7 is contemplated. In various embodiments, a population of T cells is transduced with a suicide switch as described herein, wherein at least 90% of T cells in a composition are transduced and viable and wherein the genetically modified T cells have an average vector copy number (VCN) of about 1 to 5 is contemplated.

### Methods of Treatment

Genetically-modified T cells of the invention can be used in methods for treating human subjects in need thereof and can be used to prepare medicaments for treating such subjects. The cells will usually be delivered to the recipient subject by infusion. A typical dose of T cells for the subject is between 10⁵-10⁷ cells/kg. Pediatric patients will generally receive a dose of around 10⁶ cells/kg, whereas adult patients will receive a higher dose e.g. 3×10⁶ cells/kg.

In general terms, the genetically modified T cells of the invention can be used in the same manner as known donor leukocyte infusion (DLI), but they have the added benefit of the suicide switch.

Subjects receiving the genetically-modified T cells will typically also receive other tissue from an allogeneic donor e.g. they can receive haematopoietic cells and/or haematopoietic stem cells (e.g. CD34+ cells). This allograft tissue and the genetically-modified T cells are ideally derived from the same donor, such that they will be genetically matched. Furthermore, the donor and the recipient are preferably haploidentical e.g. a matched unrelated donor, or a suitable family member. For instance, the donor may be the recipient's parent or child. Where a subject is identified as being in need of genetically-modified T cells, therefore, a suitable donor can be identified as a T cell donor.

Where genetically-modified T cells are used in conjunction with haematopoietic cells and/or haematopoietic stem cells, the genetically-modified T cells will generally be administered at a later timepoint e.g. between 20-100 days later. If the recipient develops complications after receiving the genetically-modified T cells (e.g. they develop GVHD) then the suicide switch can be triggered e.g. by administering rimiducid to the recipient.

The minimum dose of rimiducid required to eliminate the T cells will depend on the number of genetically-modified T cells which are present in the recipient. Doses above this minimum can be administered but, in accordance with normal pharmaceutical principles, excessive dosing should be avoided. We have found that a dose of 0.4 mg/kg can eliminate cells which were infused at a dose of 1.5×10⁷ cells/kg. In general terms, a rimiducid dose between 0.1-5 mg/kg is administered, and usually 0.1-2 mg/kg or 0.1-1 mg/kg will suffice, and a preferred dose is 0.4 mg/kg. A series of multiple doses of rimiducid can be administered e.g. if it is found that a first dose does not eliminate all genetically-modified T cells then a second dose can be administered, etc.

As shown herein, cells which express high levels of the cell surface transgene marker expression are more sensitive to the suicide switch trigger. These cells can also be the most problematic for GVHD. Thus, in some embodiments, a first dose of the trigger (e.g. rimiducid) is administered which kills the most sensitive cells, and then a second dose (which is higher than the first dose) is administered which kills cells which are less sensitive. Further doses (escalating where necessary) can be administered if required. Thus it is possible to deplete the problematic T cells in sequence rather than all in one go.

The recipient may undergo myeloablative conditioning prior to receiving the genetically-modified T cells (and prior to receiving an allograft). Thus the recipient's own α/β T cells (and B cells) can be depleted prior to receiving the genetically-modified T cells (and prior to receiving an allograft).

Similarly, haematopoietic (stem) cells which are administered to a recipient may be depleted for α/β cells. In contrast, genetically-modified donor T cells administered to the recipient are preferably not depleted for α/β cells.

The recipient can be a child e.g. a child aged from 0-16 years old, or from 0-10 years old. In some embodiments, the recipient is an adult.

The recipient may have a hematological cancer (such as a treatment-refractory hematological cancer) or an inherited blood disorder. For instance, the recipient may have acute lymphoblastic leukemia (ALL), acute myeloid leukemia (AML), severe combined immune-deficiency (SCID), Wiskott-Aldrich syndrome (WA), Fanconi Anemia, chronic myelogenous leukemia (CML), non-Hodgkin lymphoma (NHL), Hodgkin lymphoma (HL), or multiple myeloma.

The genetically-modified T cells may help the recipient to control transplant-related infections following receipt of an allograft.

### AOS-1

The most preferred T cell population for use with the invention are ‘AOS-1’ cells as disclosed herein. These preferred genetically modified T cells have one or more (and most preferably all) of the following characteristics:
(a) They express the iCasp9 suicide switch linked to the ΔCD19 marker, as discussed above, inserted into the genome by retroviral transduction.
(b) They have at least a 10-fold range of expression levels of the ΔCD19 marker measured by flow cytometry.
(c) There is a greater number of CD8+ T cells than CD4+ T cells.
(d) There are terminal effector memory T cells, T-effector memory cells, and T-central memory cells.
(e) Less than 60%, but at least 25%, of the T cells are naïve T cells.
(f) They were obtained from a human donor and were not subjected to a step of allodepletion.
   These cells can be obtained using the methods disclosed herein.

### General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The term “between” with reference to two values includes those two values e.g. the range “between” 10 mg and 20 mg encompasses inter alia 10, 15, and 20 mg.

The term “at least ‘X’” with reference to a number, percent, or other value is interchangeable with the term “‘X’ or more” e.g. “at least X %” is interchangeable with “X or more.”

### EXAMPLES

### Example 1: Preparation of AOS-I Cells

Di Stasi et al. (2011) supra discloses a way of preparing genetically-modified T cells with a suicide switch. AOS-1 cells were obtained by a different process, essentially as follows:
Cells are obtained from a sample of a donor's peripheral blood by leukapheresis, to enrich for white blood cells. Mononuclear cells are enriched by density gradient separation using a CliniMACS Prodigy™ apparatus (Miltenyi Biotec), and are cryopreserved for storage at −130° C.
When genetically-modified cells are to be prepared, they are thawed in a dry bath and then (day 1) cultured on the Prodigy™ apparatus in a culture bag at 37° C. and 5% CO₂. The cells are seeded into the culture medium at 1-3×10⁶ cells/mL. The medium is a serum-free medium which support T cells (containing transferrin, albumin, and insulin), supplemented with 100 U/mL IL-2, 0.2 μg/mL anti-CD3 mAb (OKT3 from Miltenyi Biotec), and 0.5 μg/mL anti-CD28 mAb. These conditions activate the T cells.
On day 3 the culture is fed with fresh 100U/mL IL-2 and 33% additional fresh medium.
On day 5 the cells are transferred to a RetroNectin™-coated AC bag and are transduced with the Gal-V retroviral vector encoding the iCasp9 suicide switch disclosed by Tey et al. (2007) supra and Di Stasi et al. (2011) supra. IL-2 is added at 200U/mL.
On day 6 the cells are processed to remove remaining retroviral particles (supernatant), and are re-suspended in medium at 10⁶ cells/mL with 200U/mL IL-2. Culture continues at 37° C., 5% CO₂.
On day 8 transduced cells are selected by MACS on the basis of CD19 expression, again using the Prodigy™ system. The CD19+ cells are cultured in a bag, as before, with further IL-2 (200 IU/mL) at 10⁶ cells/mL.
On day 9 the cells are harvested, washed with PlasmaLyte A (Rizoli (2011) J Trauma 70(5 Suppl) S17-8) in a centrifuge, and resuspended in cryopreservation medium for storage at −130° C. until they are needed by an allogeneic recipient.
If, however, cell numbers were not adequate at day 9 (e.g. too few cells for the intended treatment) then culture can be continued up to day 15 prior to harvest.

Thus a key difference compared to Di Stasi et al. (2011) supra is that the cells are activated much earlier e.g. IL-2 and anti-CD3 are added to the culture on day 1, rather than day 5. Moreover, anti-CD28 antibodies are employed on day 1.

Cells obtained by this process have a much lower CD4+/CD8+ ratio than the initial donor cells (changing from a majority of CD4+ cells to a majority of CD8+ cells), so the process enriches for CD8+ cells T cells relative to CD4+ T cells. Moreover, they have been found to give rise to GVHD in allograft recipients less frequently than observed in previous in vivo studies. Without wishing to be bound by theory, this reduced risk could be because of the reported link between GVHD and CD4+ cells (e.g. see Coghill et al. (2011) Blood 117:3268-76) and/or because the process for preparation of the T cells involves expansion in vitro which limits their potential for further expansion in vivo.

### Example 2: Differential Expression of iC9 in Allogeneic T Cells Allows Selective Depletion of Activated T Cells Following Exposure to Rimiducid and Permits In Vivo Allodepletion

AOS-1 cells prepared in Example 1 can provide virus and tumor-specific immunity following stem cell transplant. In instances of GVHD, activation of iC9 with rimiducid leads to rapid killing of alloreactive T cells and resolution of GVHD. However, gene-modified T cells can re-expand in the host. This example evaluates T cell subsets and the relationship between transgene expression and rimiducid sensitivity, in order to understand differential apoptosis in patients treated with allogeneic iC9-modified T cells.

The results show that in vivo depletion of T cells with iC9 is dependent on the level of transgene expression, which is regulated by the activation state of the T cell. Highly activated alloreactive T cells express higher levels of iC9 which makes them more sensitive to rimiducid-induced apoptosis, and serves to selectively deplete GvHD-causing T cells while sparing the host's own T cells. These results show that sufficient iC9 transgene expression is a key element to trigger the apoptosis induced by rimiducid. It was also observed that apoptosis correlated with the intensity of iC9 expression.

Complete dimerizer-dependent elimination could be achieved by the selection of highest transgene expressing cells. Elimination could be enhanced by T-cell activation. iC9 transgene expression could be upregulated by reactivation in vitro, thus enabling triggering of apoptosis in the presence of rimiducid. The low iC9-bearing population has greater potency to be eliminated upon TCR activation. This indicates the possibility of further elimination by rimiducid when AOS-1 is highly activated.

### Plasmid, Retrovirus, and Retroviral Transduction

The safety switch system is a mutated FKBP12 binding protein linked to caspase-9 and truncated CD19 (ΔCD19) to allow selection of gene-modified T cells (SFG-iC9-ΔCD19). Exposure to rimiducid causes dimerization of the protein, resulting in apoptosis of the modified T cells. SFG-iC9-ΔCD19 has been previously described (Di Stasi et al. (2011) supra; Tey et al. (2007) supra), along with methods for T cell activation & expansion, and for retroviral transduction to introduce the SFG-iC9-ΔCD19 construct.

### Immunophenotyping, Spectratyping, Western Blot and PCR

To evaluate the effect of transgene expression levels to the sensitivity of rimiducid-induced apoptosis, AOS-1 T cells were stained with anti-CD19 and anti-CD3, and sorted into 3 equal populations based on the intensity of CD19 staining (CD19^{high}, CD19^{med} and CD19^{low}). To measure apoptosis 5×10⁵ cells were plated per 48-well, and rimiducid was added at a concentration of 10 nM in a 4-hour assay. Phenotyping and functional assays (e.g., apoptosis) were performed by flow cytometry, qPCR and Western blot before and after T cell reactivation using anti-CD3/anti-CD28 antibodies. The phenotype of the cells was determined by staining cells for flow cytometry analysis with anti-CD3, anti-CD4, anti-CD8, anti-CD19, anti-CD45RA, anti-CD45RO, and anti-CCR7 antibodies. Terminal effector memory cells are defined as CD45RA⁺CD45RO⁻CCR7⁻. T effector memory cells are defined as CD45RA⁻CD45RO⁺CCR7⁻. T central memory cells are defined as CD45RA⁻CD45RO⁺CCR7⁺. Naïve T cells are defined as CD45RA⁺CD45RO⁻CCR7⁺. Diversity of the T cell receptor repertoire was assessed by flow cytometry.

### In Vivo Studies

In vivo studies were performed by i.v. injection of control or gene-modified T cells co-expressing luciferase into NSG mice. 1×10⁷ EGFPluc+ iC9-T cells were infused into NSG mice. T cell bioluminescence was measured at 2 and 24 hours after infusion, followed by i.p. injection of a titrated dose of rimiducid or placebo (control drug). The animals were administered either control drug (temsirolimus; 1 mg/kg), rimiducid (0.001-1 mg/kg), or vehicle. Remaining T cell bioluminescence was measured at 6, 24 and 48 hours after drug administration. Bioluminescent imaging and flow cytometry were performed to assess in vivo depletion following iC9 activation.

### Results

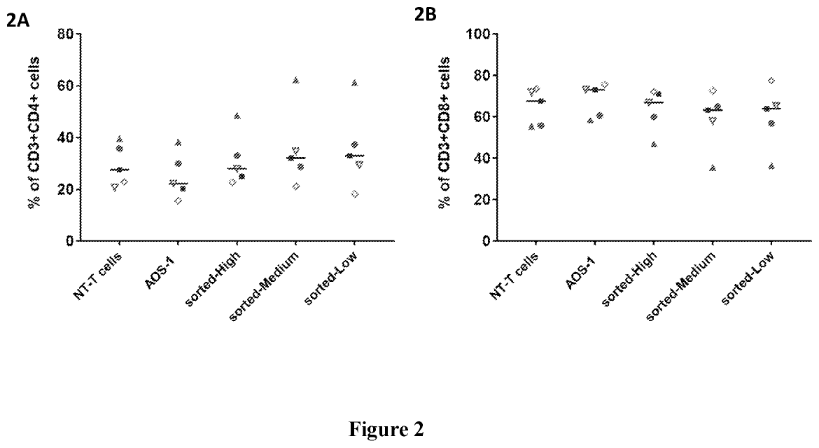

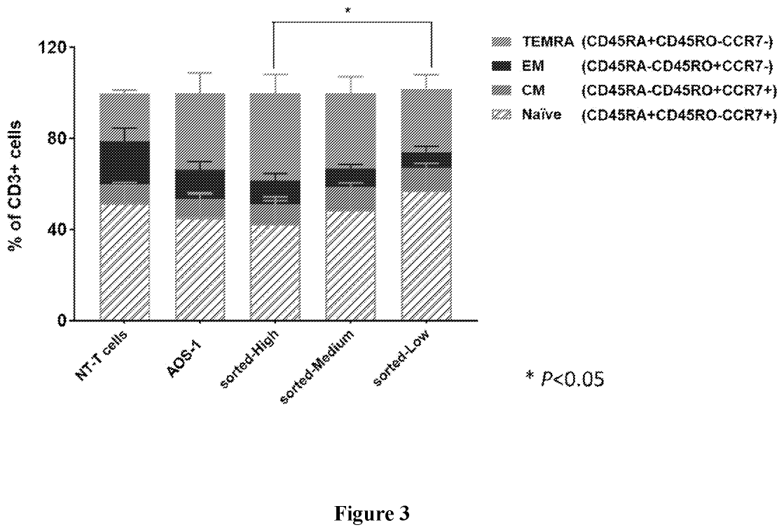

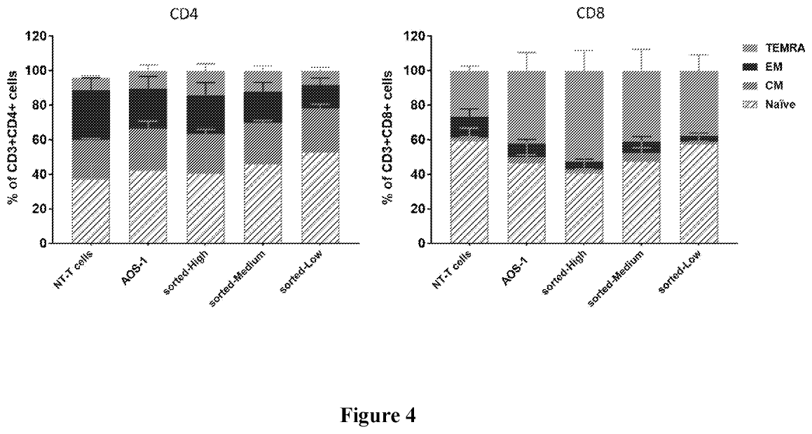

Purity of AOS-1 cells after transduction and CD19 selection was 95% (FIG. 1A). Sorting AOS-1 cells based on CD19 mean fluorescence intensity (MFI) resulted in a CD19 MFI of 73, 46 and 22 for CD19 high, medium and low sorted populations, respectively (FIG. 1B). FIG. 2 shows that there was no significant difference in the % of CD8 and CD4 compartments among these cells. FIG. 3 shows that iC9-ΔCD19^{low} expressing cells contained fewer terminal effector memory (TEMRA) cells and more naïve-like cells than iC9-ΔCD19^{high} (28+6% vs. 39+8% (TEMRA), and 57+9% vs. 42±12% (naïve), respectively, P<0.05). This was consistent between CD4+ and CD8+ subsets (FIG. 4).

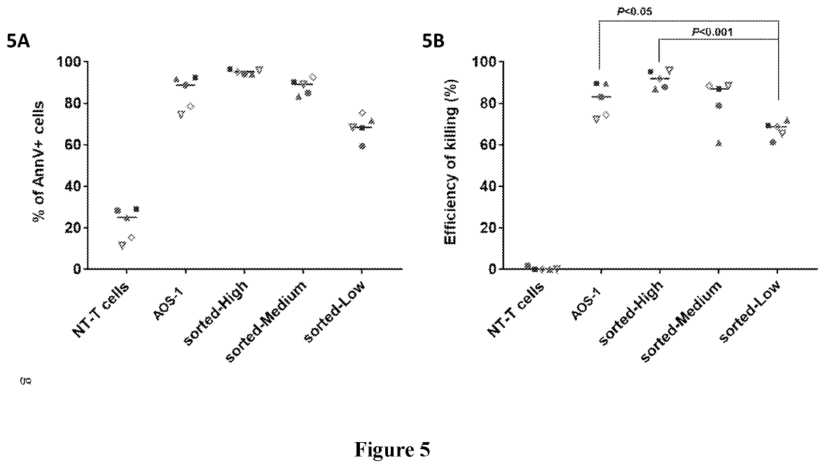

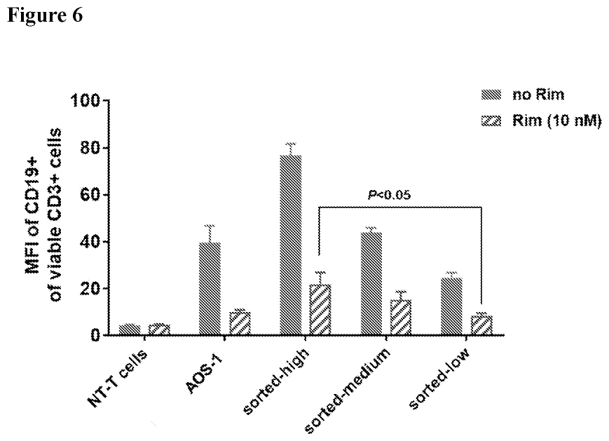

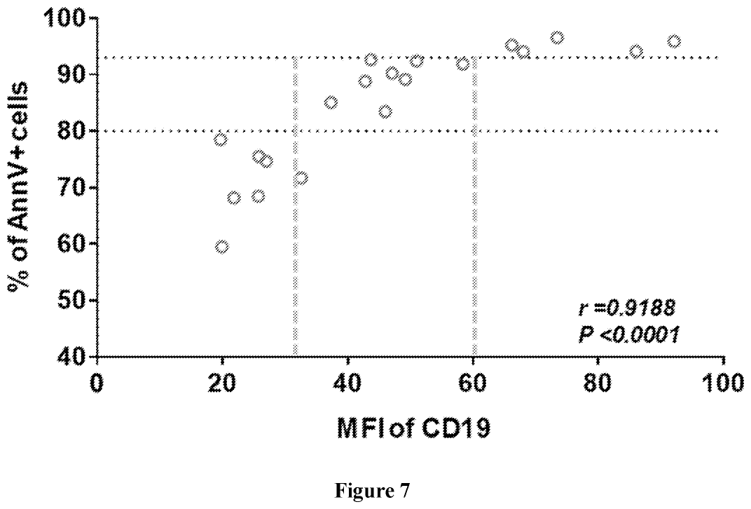

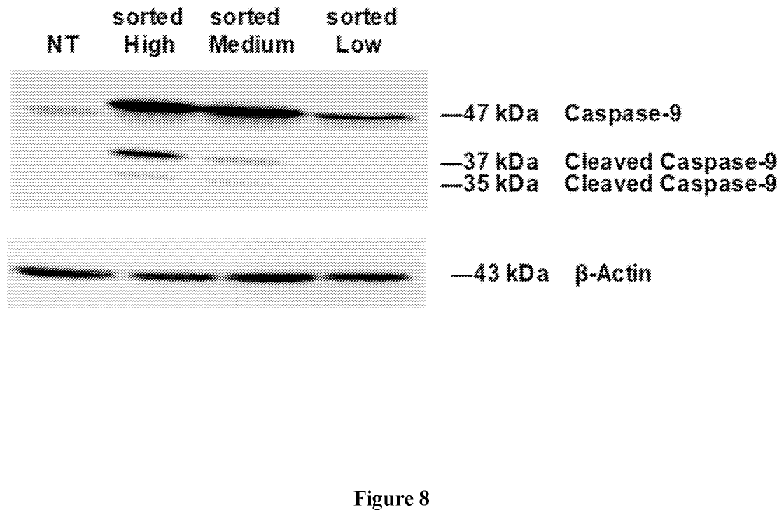

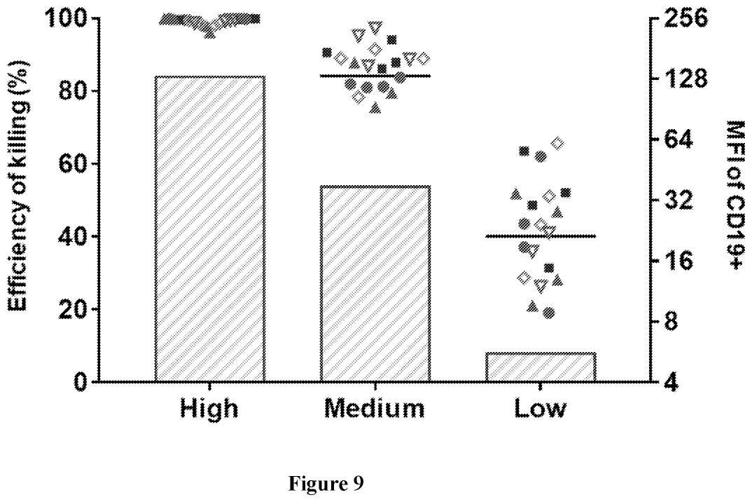

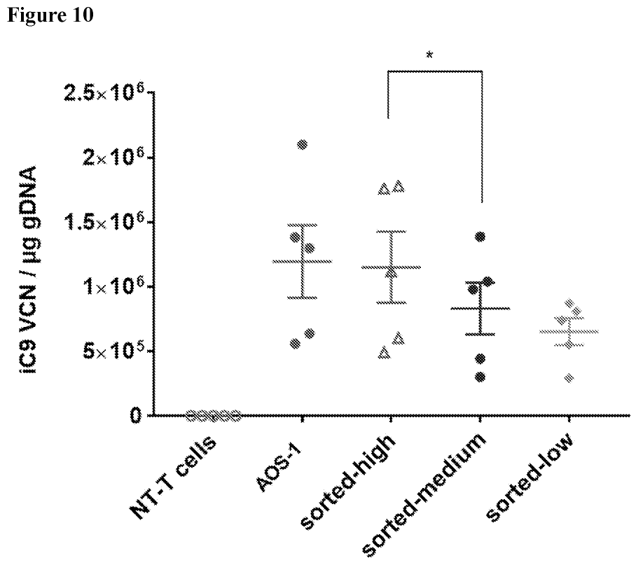

In a 4-hour apoptosis assay, the killing efficiency was significantly diminished in iC9-ΔCD19^{low} compared to unsorted or iC9-ΔCD19^{high} cells (P<0.05, P<0.001, respectively). FIG. 5 shows that more than 95% of high MFI of CD19 cells has gone through apoptosis within 4 hours, and the median killing efficacy is 92%, 87% and 69% in sorted high, medium and low cells. FIGS. 6 & 7 show that apoptosis is correlated with the intensity of the iC9-ΔCD19 transgene expression. FIG. 8 shows that iC9-ΔCD19^{low} cells also expressed less caspase-9 protein as measured by Western blot, correlating the levels of caspase-9 protein to decreased rimiducid sensitivity. Thus, sufficient expression of iC9 is the prerequisite to trigger the apoptosis. The low CD19-expressing population has lower caspase-9 protein, indicating that the CD19 expression is correlated with iC9 protein level, which is the key element to trigger apoptosis. FIG. 9 shows that apoptosis is correlated with the intensity of the iC9-ΔCD19 transgene expression. In addition, there is a statistical difference between high and medium CD19+ populations regarding to vector copy number (VCN) per μg of genomic DNA (*P=0.047), iC9-expressing cells with higher CD19 MFI seems have a greater VCN, as shown in FIG. 10, which shows VCN per μg of genomic DNA measured by qPCR in the different cell subsets.

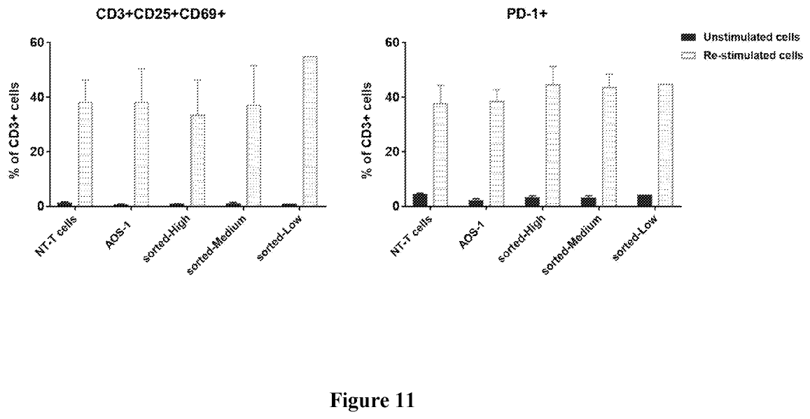

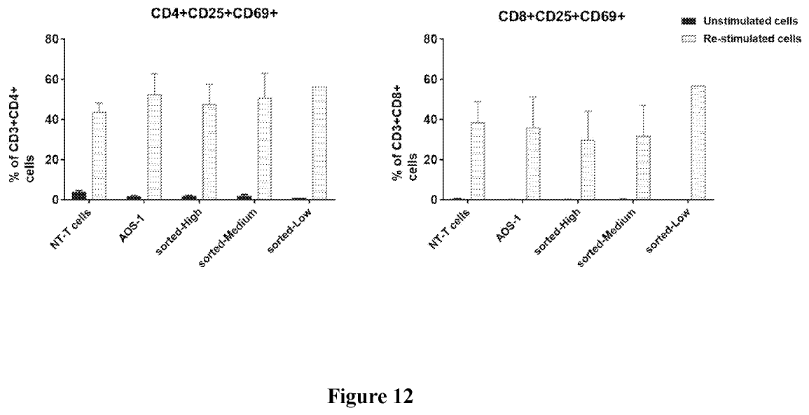

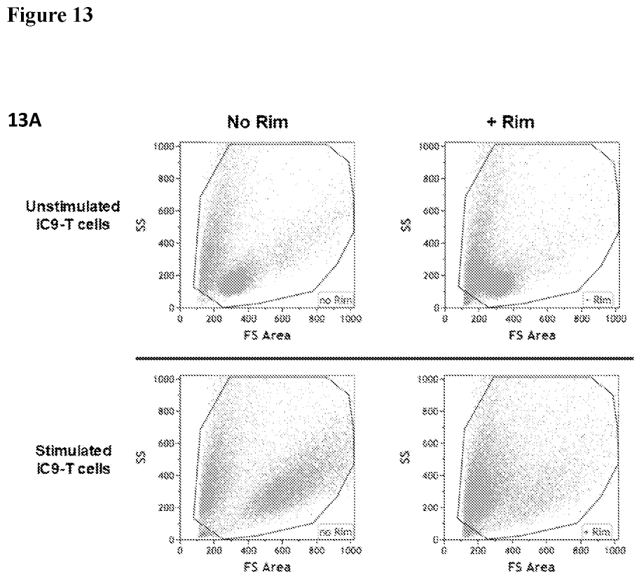

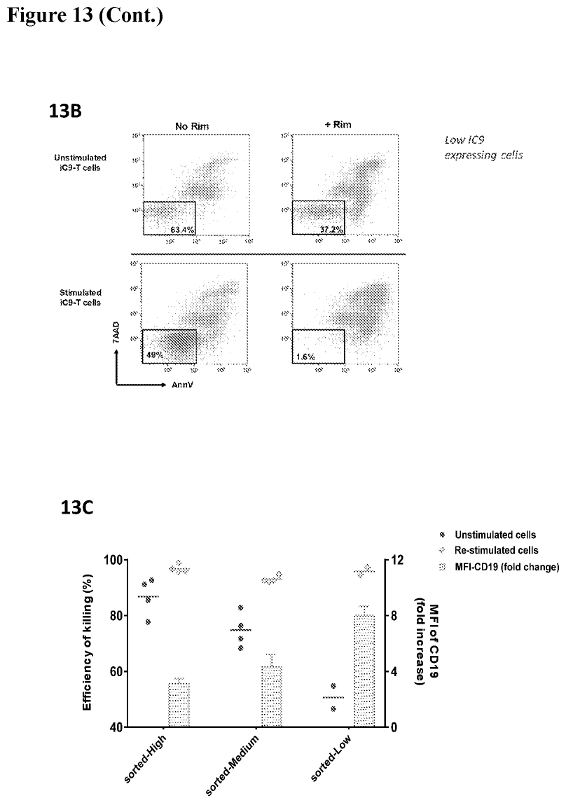

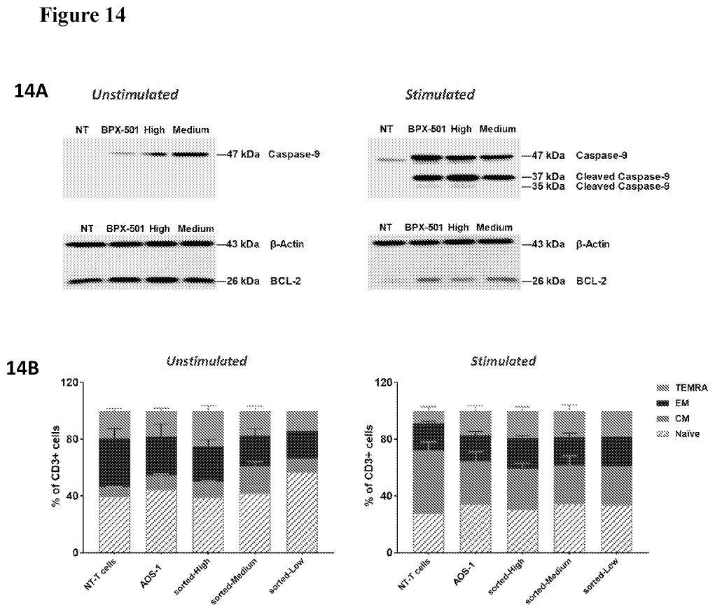

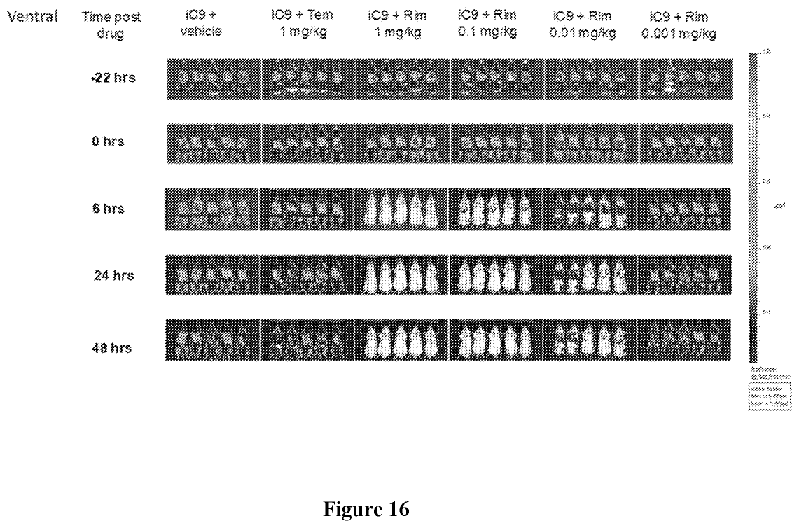

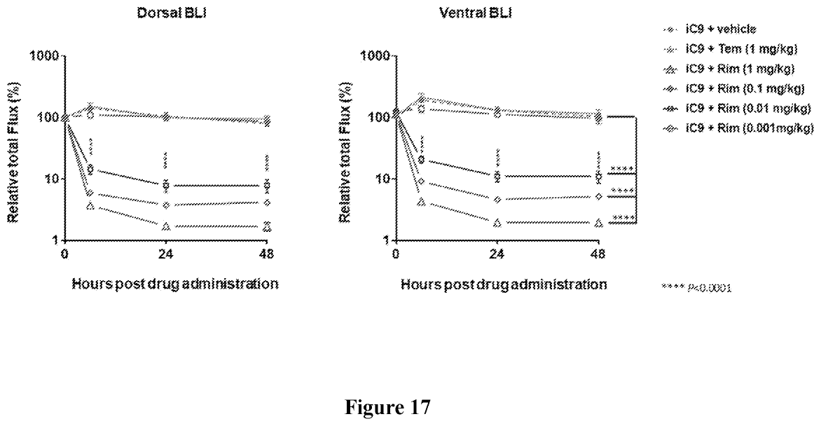

Animal studies showed a dose dependent decrease in the MFI of CD19 even in mice treated with 0.001 mg/kg rimiducid demonstrating preferential killing of iC9-ΔCD19^{high} T cells, and sparing of iC9-ΔCD191^{low} T cells (FIGS. 16-18). However, because iC9-ΔCD19 transgene expression is regulated by the retroviral LTR promoter and sensitive to the activation state of the T cell, we measured the activation status following TCR cross-linking using CD25, CD29 and PD-1 markers, and transgene levels using CD19. FIG. 11 shows up-regulation of CD25, CD69, and PD-1 on CD3+ T cells upon ex vivo stimulation. FIG. 12 shows up-regulation of CD25, CD69, and PD-1 on CD3+CD4+ and CD3+CD8+ T cells upon ex vivo stimulation. FIGS. 13A-C show that while unstimulated iC9-ΔCD19 showed differential killing based on transgene expression (86%, 76% and 50% for high, medium and low iC9-ΔCD19, respectively), reactivation increased transgene MFI and apoptosis in all fractions to over 90% when exposed to rimiducid. In low-CD19 sorted cells, CD19+ specific killing was increased from 46% to 97% after cells were activated in vitro (FIGS. 13A & 13B), and the mean MFI of CD19 increased 8 fold (FIG. 13C). This is consistent with caspase-9 protein level, transgene expression could be upregulated by reactivation in vitro, which enables triggering of apoptosis in the presence of rimiducid (FIG. 14A). FIG. 14A also shows that the increase in iC9 levels when cells were activated correlates with decreased expression of anti-apoptotic protein BCL-2. FIG. 14B shows the transition of naïve and memory populations upon activation. Taken together, these results confirm the relationship of T cell activation with transgene expression.

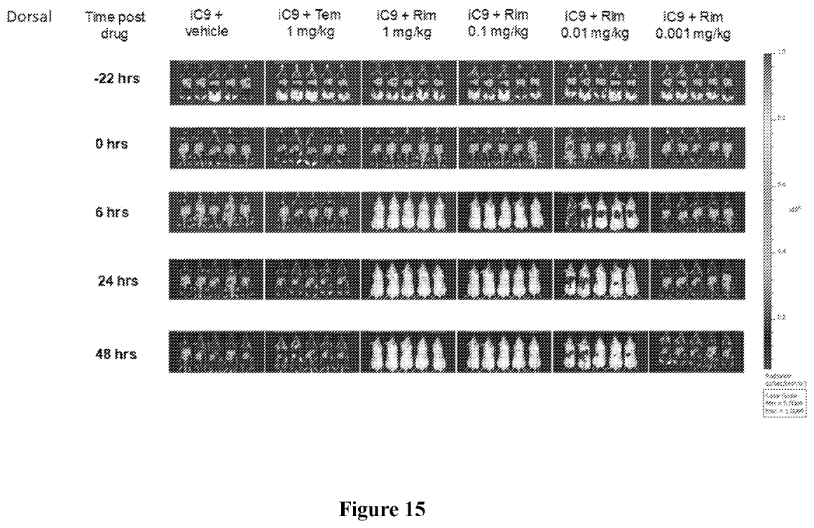

FIGS. 15-17 show that iC9-EGFP T cells were efficiently eliminated at 6 hours post drug administration, and cells were further depleted at 24 hours and 48 hours in low dose group (0.01 mg/kg). There is significant decrease of BLI in mice given 1 mg/kg, 0.1 mg/kg, and 0.01 mg/kg at 24 hours and 48 hours post treatment (P<0.001). The relative total flux of iRC9-T cells in mice given 1 mg/kg of rimiducid, reduced 96%, 98.1% and 98.1% at 6-hour, 24-hour and 48-hour, respectively. A reduction of 92.5%, 95.5% and 95.3% was observed at 6-hour, 24-hour and 48-hour in mice treated with 0.1 mg/kg rimiducid, respectively. And a reduction of 82.5%, 90.5% and 90.5% was observed at 6-hour, 24-hour and 48-hour in those mice treated with 0.01 mg/kg rimiducid, respectively (P<0.001).

The maximal elimination effect in the kill switch induced by rimiducid was reached 24 hours post drug administration. The values for average radiance were normalized to the initial radiance in each mouse using the formula: D Average radiance=100*[Timepoint “X” radiance]/[Timepoint “0” radiance]. Error bars represents S.E.M. Two-way ANOVA test was used to assess statistical significance of differences in T cell radiance between multiple groups at different time points.

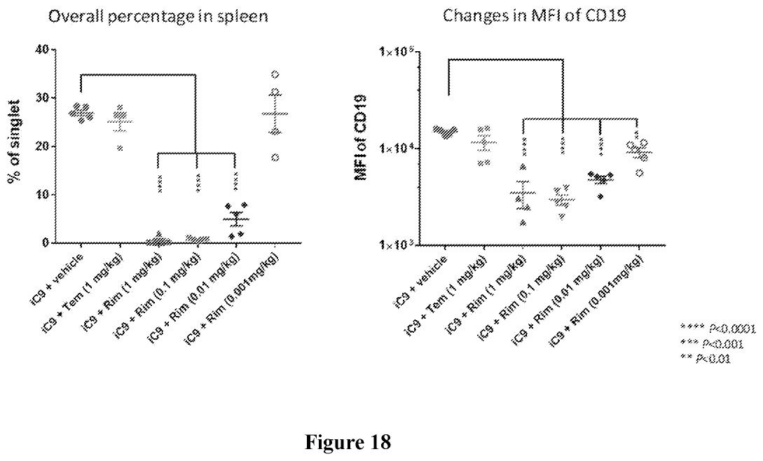

FIG. 18 shows that iC9 selectively eliminated the most highly expressing and most highly transduced T cells. FIG. 18 depicts a flow cytometric analysis of cells harvested from spleens 48 h post drug treatment. The left panel demonstrates that CD3+CD19+ cells (iC9+ T cells) were significantly eliminated after treated with 0.01 mg/kg, 0.1 mg/kg and 1 mg/kg Rim, compared to the vehicle group (p<0.001). A reduction of 99.6%, 98.6% and 89.6% was observed in each group, respectively. The right panel of FIG. 18 shows the MFI of CD19 in CD3+CD19+ cells. Consistently with previous data, a dose dependent decrease in the MFI of CD19 was observed in each of the treated groups.

### Example 3: Characterization of Allogeneic T Cells Expressing ic9 Following Adoptive Transfer in Children Receiving a Haploidentical Stem Cell Transplant for the Treatment of Myeloid Malignancies

Adoptive transfer of allogeneic donor T cells can be an effective treatment for hematological malignancies through recognition of leukemia-associated antigens (LAAs) on tumor cells or through alloreactivity. However, alloreactive T cells can also cause GvHD, limiting their use as an immunotherapy. To leverage the anti-tumor effects of allogeneic T cells while minimizing GvHD, we have genetically modified donor T cells with the iC9 safety switch, which induces apoptosis following exposure to rimiducid. Here we show that these AOS-1 cells persist, expand and contain functional LAA-specific T cells in children receiving an α/β TCR and CD19-depleted haploidentical HSCT for the treatment of myeloid malignancies.

The results show that allogeneic AOS-1 T cells engineered with the iC9 safety switch engraft, expand and demonstrate long-term persistence following adoptive transfer into patients receiving a haplo-HSCT. LAA-specific T cells and alloreactive T cells within the AOS-1 product are detectable in the peripheral blood following infusion and likely contribute to elimination of myeloid malignancies. Moreover, cells which expressed high levels of the CD19 transgene marker were shown to be eliminated more rapidly in vivo than cells which express low levels.

### Methods

Pre-infusion products (AOS-1: donor T cells modified with the bicistronic retroviral vector encoding iC9 and truncated CD19 (ΔCD19)) and patient peripheral blood mononuclear cells (PBMCs) were analyzed from twelve patients (AML (10), MDS (1), JMML (1)) receiving AOS-1 (1×10⁶ cells/kg) following an α/β T cell and CD19 B cell-depleted haplo-HSCT. T cells modified with the retroviral vector encoding iC9 and ΔCD19 were prepared as described in Example 1.

Engraftment and persistence of the donor modified T cells were measured by co-expression of CD3 and CD19 by flow cytometry. Endogenous and gene-modified T cells were also phenotyped for CD4:CD8 ratios, memory cell composition (TN, TCM, TEM, TEMRA; CD45RA and CD62L) and T cell receptor Vβ diversity. AOS-1 products and post-treatment samples were characterized for LAA-specific T cells using IFN-γ ELIspot against peptide pools (15 aa overlapping by 5 aa) derived from WT1, PRAME, MAGE (A1, C1, C3), NE and PR3, with and without exposure to 10 nM rimiducid to determine the anti-leukemic contribution of AOS-1.

### Cell Preparation

PBMCs were mixed with pre-warmed (37° C.) cell culture media and centrifuged cells at 400×g rpm for 10′ at 4° C. Cells were resuspended and counted using Cellometer. Cells were then centrifuged again at 1200 rpm for 10′. The cells were then resuspended in culture media at a concentration of 1×10⁶/mL and 100 μL were added per well.

### Preparation of Peptides and PHA

100 μL of 2X peptide stock or PHA were added to the desired wells. The plates were mixed gently for 2′. The plates were transferred to a 37° C. incubator with 5% CO₂ and incubated for 18-24 hours.

### ELISpot Plate Preparation and Development

100 μL capture anti-IFNγ antibody was added to each PBS-pre wetted well. The plates were then incubated at 4±3° C. overnight. After incubation the plates were washed 3 times with 200 μL D-PBS. Unbound sites were blocked with 100 μL D-PBS containing 10% culture media for 120+15 minutes at room temperature in a BSC. The plates were then washed 3 times with 200 μL D-PBS. 200 μL D-PBS were added into the wells. The plates were left at room temperature until the cells are ready.

When cells were ready plates were removed from the incubator and the wells rinsed 2 times with tap water. The plates were then washed 5 times with D-PBS, containing 0.05% Tween 20. The wells and well bottoms were rinsed with distilled water. 100 μL 1 μg/ml biotin-anti-IFNγ antibody were added and incubated in a 37° C. incubator for 1 hour. The plates were then washed as described before. 100 μL Extravidin-Alkaline-Phosphatase were added to each well and incubated for 75′. Plates were then washed with tap water, wash buffer, and with distilled water. 100 μL of BCIP/NBT substrate solution was added to each well and incubated for 5-10 minutes at room temperature. The reaction was stopped by rinsing the plate with tap water. Plates were left to dry at room temperature before reading on an ELISpot reader.

### Results

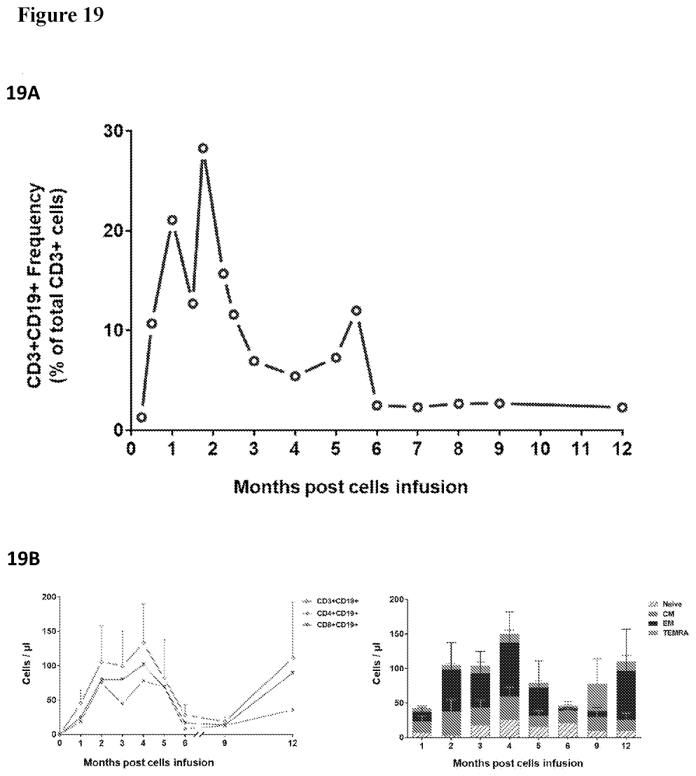

AOS-1 was infused at a median time of 22.5 days after HSCT (range 12-34, one patient was infused at day 89 and one patient was infused at day 147). AOS-1 cells (CD3+CD19+) were detectable in peripheral blood 1-2 weeks after infusion in all 12 patients, reaching a peak expansion frequency of a median of 24±17% of total CD3+ T cells, and an absolute cell number of 66.9+35.6 cells/μL at 2 months post-infusion and could be detected for up to 24 months (FIG. 19A).

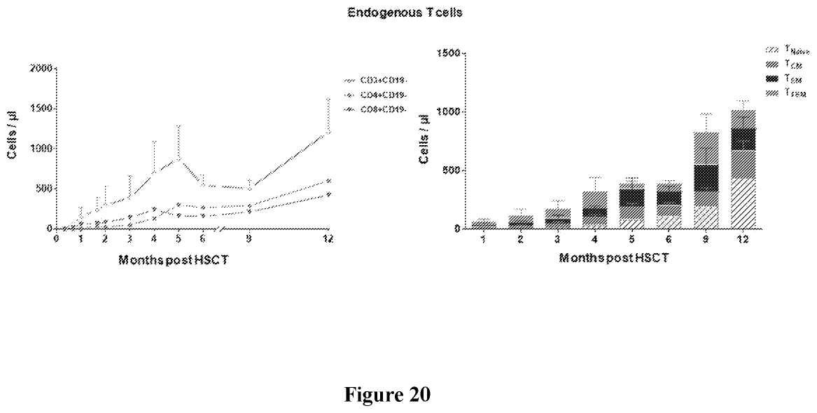

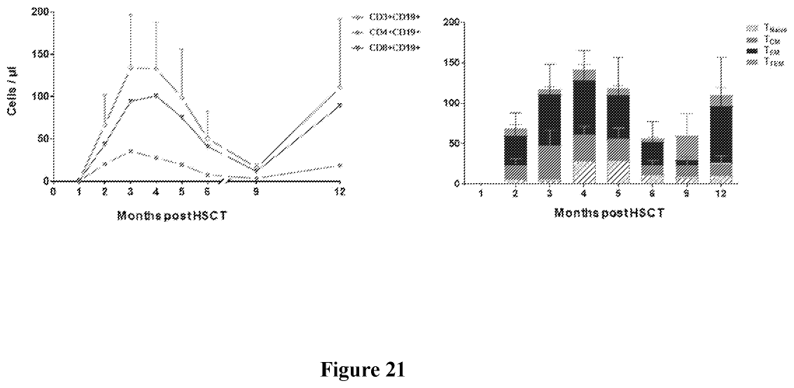

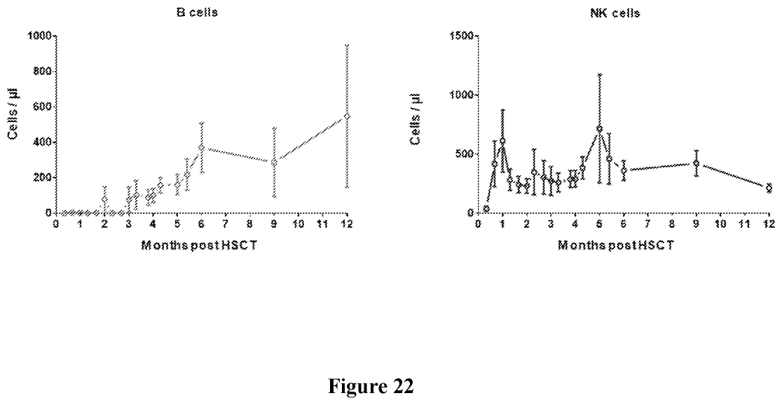

AOS-1 T cells showed a CD8-skewed phenotype whereas endogenous T cells exhibited a more balanced CD4:CD8 ratio (FIGS. 19B, 20 & 21). The maximum absolute number of AOS-1 cells was reached at 4 month post AOS-1 infusion. AOS-1 were predominantly CD45RA−CD62L+ and CD45RA−CD62L− central and effector memory T cells, respectively (FIG. 19B). FIG. 20 shows that the mean absolute count of CD3+CD19− T cells were greater than 697+396 cells/μL at 4 month post HSCT, and the mean absolute count of CD4+CD19− T cells is 301±77 per μL at 5 months post HSCT. The ratios of CD4:CD8 CD3+CD19− T were 1.85, 1.67, and 1.4 at month 5, 6, and 12, respectively. In line with the time frame of endogenous T cells reconstitution, the maximum expansion of absolute AOS-1 cells reached at 3-4 month post HSCT where the endogenous T cells is still under recovery, the beneficial effects of anti-viral and anti-tumor immunity from AOS-1 cells are more important to protect viral infections and relapse (FIG. 21). FIG. 22 shows the reconstitution of endogenous NK and B cells post HSCT.

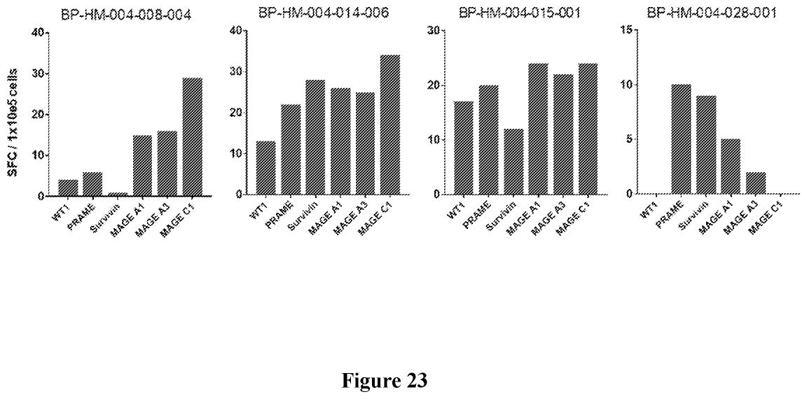

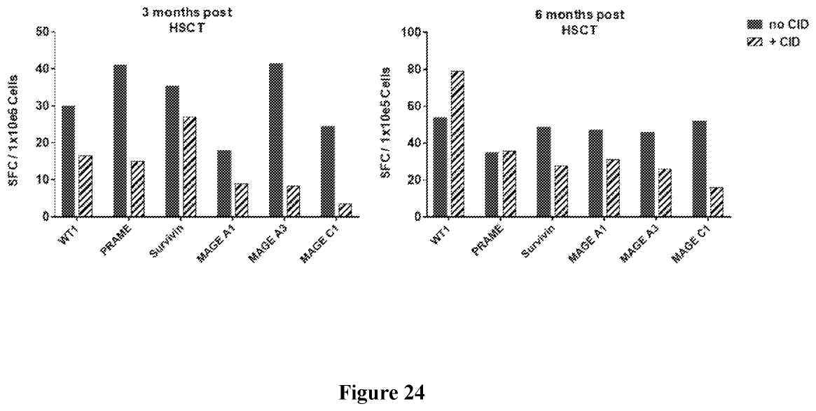

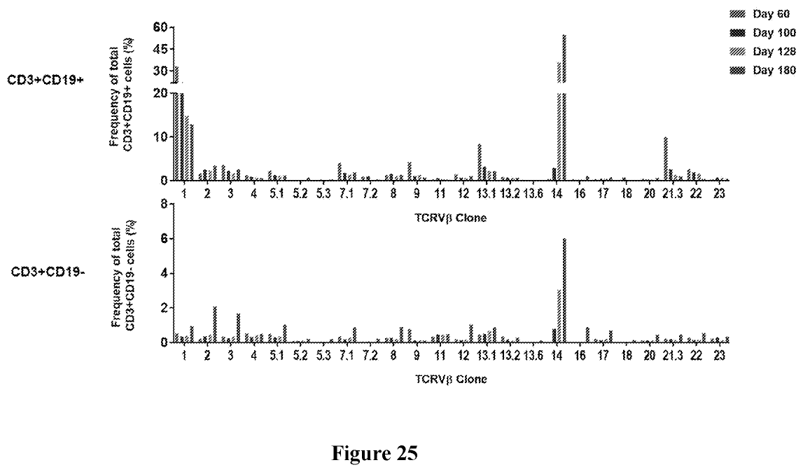

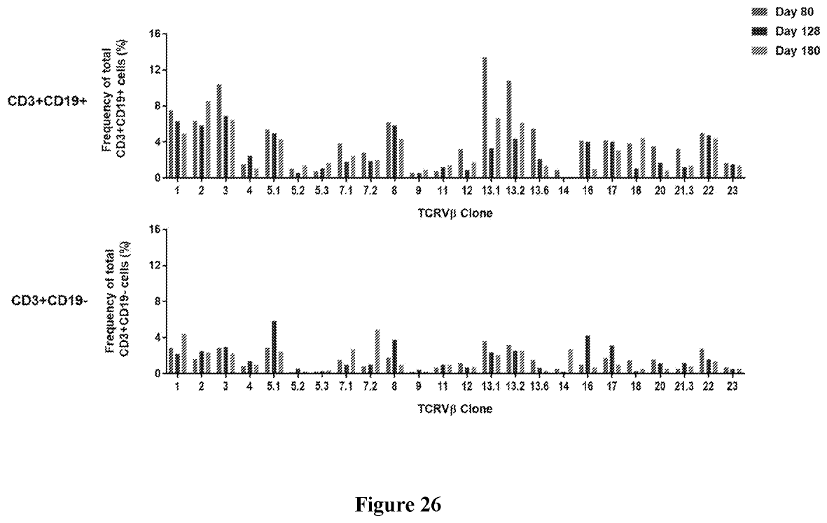

In AOS-1 products, we detected LAA-specific T cells by ELIspot using overlapping peptide pools to WT1, PRAME, MAGE, NE and PR3, and in peripheral blood samples obtained 2-5 months post-T cell infusion (FIG. 23). Importantly, LAA-reactivity was greatly diminished with exposure to iC9-activating rimiducid (FIG. 23). Further, the TCR VP usage was measured and a highly-skewed TCR repertoire in AOS-1 T cells was observed compared to endogenous T cells 6 months after HSCT indicating selection and expansion of TCR clones (FIGS. 25 & 26). Three patients engrafted with AOS-1 were treated with rimiducid to control GvHD resulting in a rapid decrease (62±12%) of CD3+CD19+ T cells in the peripheral blood. In patients treated with rimiducid, CD3+CD19+ T cells recovered without further instances of GvHD suggestive of in vivo depletion of alloreactive T cell clones using iC9.

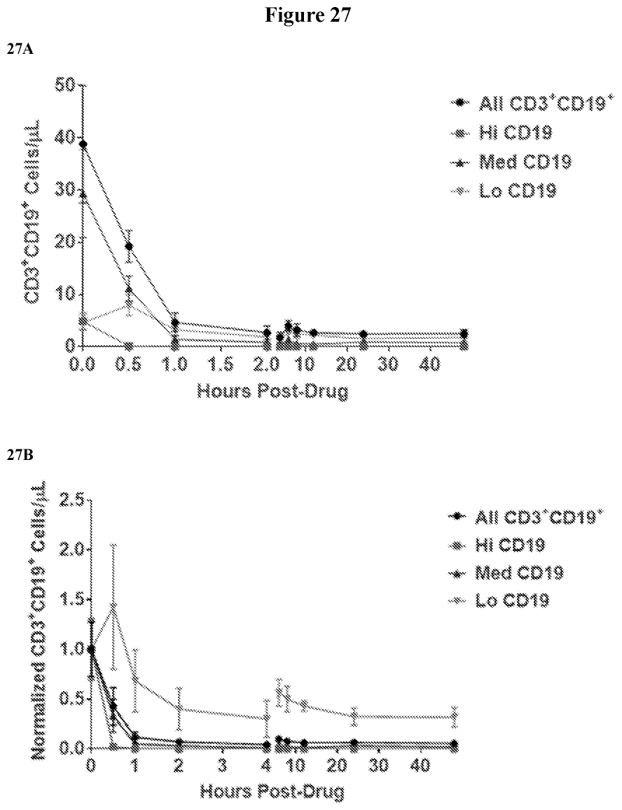

Further experiments looked at the speed of elimination of low-CD19, medium-CD19 and high-CD19 populations after administration of rimiducid. FIG. 27 shows that: CD19-high cells (▪) were eliminated within half an hour; levels of CD19-medium cells (▴) fell to near-zero within an hour; but levels of CD19-low cells (▾) declined more slowly. This effect was seen for both absolute numbers of cells (FIG. 27A) and for numbers normalised according to the starting concentration for each separate population (FIG. 27B).

### Example 4: Vector Copy Number (VCN) Analysis

Vector copy numbers (VCN), one of the critical quality attributes, from clinical manufacturing of cell therapy products were evaluated for AOS-1 cells prepared as described in Example 1.

### Methods

Multiplex qPCR assay was designed to detect the cell product-specific transgene transduced as described in Example 1 and a reference gene. Relative ΔΔCT method is employed in combination with a characterized calibrator to translate the sample CT values into vector copy number per cell.

The assay was executed in 96-well plate format on an Applied Biosystems 7500 Fast Real-Time PCR System, using qualified primers/probe combinations for simultaneous amplification of a cell product-specific transgene using iCaspase9-specific, FAM-labelled and a reference TFRC gene using HEX-labelled TaqMan probe in a duplex assay. All qPCR samples were prepared and transferred into the qPCR 96-well reaction plate inside a PCR workstation or a BSC.

DNA isolated from untransduced and transduced samples is analyzed by a multiplex qPCR assay in 6 replicates using a qualified transgene primer/probe combination designed for detection of iCaspase9, and TFRC reference gene. DNA isolated from characterized calibrator cells, which have a known transgene copy number, is analyzed together with PC and TA samples and serves as a reference point for VCN calculation. Each qPCR reaction contains 5 ng of sample DNA. DNA isolated from PC sample serves as a negative control for the qPCR assay.

### Results

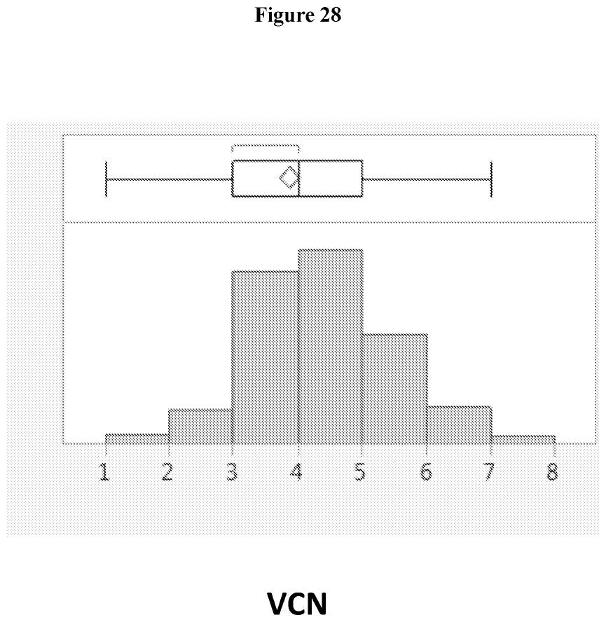

VCN numbers for AOS-1 cells prepared as described in Example 1 are shown in distribution (FIG. 28). The near normal distribution (normal 2 mixture) has a mean close to 4.0 with a range from 1 to 7. With regards to variation, it is a 3 Standard Deviation distribution which means, in a normal distribution case, 99.7% of values drawn from the distribution are within three standard deviations.

There is no outlier identified.

### Example 5: IFNγ Potency of AOS-1 Cells

The functionality of these AOS-1 cells prepared as described in Example 1 was measured via different IFNγ secretion upon stimulation with with anti-CD3. The data of this study demonstrates that transduction of donor T cells with the caspase 9 retrovirus vector does not alter their ability to provide immunity against opportunistic infections. This also suggests the normality of function of the T cells contained within AOS-1 cells.

### Methods

T cells obtained as described in Example 1 were resuspended and activated in vitro using antibodies against CD3 for 24 hours.

The cell culture supernatant containing the secreted cytokines was frozen at −20° C. until ready to be assayed to determine the cytokine concentration using ELISA. Both assays were carried out as per manufacturer's recommendations.

Coming's anti-CD3 antibody pre-coated 96-well format were used for T cell activation. The plates are coated with 2-8 μg/cm2 (or 0.64-2.56 μg/well) of anti-CD3 antibody (Clone UCHT1).

Two different AOS-1 cell samples (1A and 1B) were seeded at 200 μL per well at different seeding densities of 1×10⁶, 0.5×10⁶, 0.25×10⁶, 0.125×10⁶ and 0.06×10⁶ per mL and activated for 24 hr. The supernatant containing secreted IFNγ was collected and transferred to fresh 96-well tissue culture treated plate and frozen down at −20° C. for at least one day. The collected supernatant was thawed and assayed by ELISA for quantifying the amount of IFNγ in the cell culture supernatant.

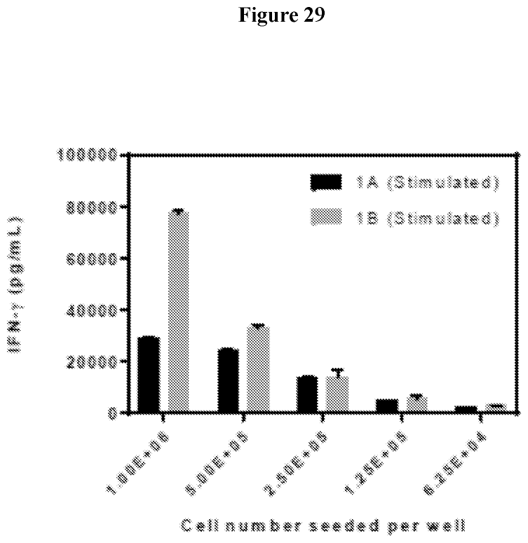

The BioCoat plate elicited a potent response in the cells and shows a response that is dependent on the number of cells seeded (FIG. 29).

### Example 6: Representative Embodiments

A1. A composition comprising genetically-modified T cells comprising genetically-modified CD4⁺ T cells and genetically-modified CD8⁺ T cells, wherein: (i) the genetically-modified T cells express a suicide switch; and (ii) about 25% to 60% of the genetically-modified T cells are naïve T cells.

A2. The composition of embodiment A1 wherein about 30-60% of the genetically-modified T cells are naïve T cells.

A3. The composition of embodiment A1 wherein about 42-49% of the genetically-modified T cells are naïve T cells.

A4. The composition of embodiment A1, wherein the ratio of genetically-modified CD4+ T cells to genetically-modified CD8+ T cells in the composition is less than 2.

A5. The composition of any of the preceding embodiments, wherein at least 10% of the genetically-modified CD8+ T cells are terminal effector memory T cells and/or no more than 58% of the genetically-modified CD8+ T cells are naïve T cells.

A6. The composition of embodiment A5, wherein at least 30% of the genetically-modified CD8+ T cells are terminal effector memory T cells and no more than 50% of the genetically-modified CD8+ T cells are naïve T cells.

A7. The composition of any of the preceding embodiments, wherein the genetically-modified T cells display a range of expression levels of the cell surface transgene marker, wherein the range is at least 10-fold, wherein the expression levels are measured by flow cytometry, and wherein the expression levels are measured as mean fluorescence intensity (MFI) values.

A8. The composition of embodiment A7, wherein the range is at least 100-fold.

A9. The composition of embodiment A7 or A8, wherein the genetically-modified T cells display a range of sensitivities to a trigger molecule, such that exposure of the cells to a particular concentration of the trigger molecule leads to death of at least 10% of the cells but permits at least 10% of the cells to survive.

A 10. The composition of any preceding embodiment, wherein the ratio of genetically-modified CD4+ T cells to genetically-modified CD8+ T cells in the composition is less than 0.5.

A11. The composition of any preceding embodiment, wherein the suicide switch comprises caspase-9.

A12. The composition of any preceding embodiment, wherein the suicide switch comprises a FKBP12 region, a FKBP12 variant region, a FKBP12-Rapamycin Binding (FRB) or FRB variant region

A13. The composition of any one of embodiments A9-A12, wherein the trigger molecule is rapamycin, a rapalog, AP1903, AP20187, or AP1510.

A14. The composition of embodiment A12, wherein the FKBP12 variant region has an amino acid substitution at position 36 selected from the group consisting of valine, leucine, isoleuceine and alanine.

A15. The composition of embodiment A13, wherein the FKBP12 variant region comprises two copies of FKBP12v36.

A16. The composition of any preceding embodiment, wherein the genetically-modified T cells are human T cells.

A17. The composition of any preceding embodiment, wherein: (i) the genetically-modified T cells express the iCasp9 suicide switch linked to the ΔCD19 marker, inserted into the genome of the T cells by retroviral transduction; (ii) the genetically-modified T cells have at least a 10-fold range of expression levels of a ΔCD19 cell surface marker measured by flow cytometry; (iii) the composition includes a greater number of CD8+ the genetically-modified T cells than CD4+ the genetically-modified T cells; (iv) the composition includes genetically-modified terminal effector memory T cells, genetically-modified T effector memory cells, and genetically-modified T central memory cells; (v) less than 50% of the genetically-modified T cells are naïve T cells; and (vi) the T cells were obtained from a human donor and were not subjected to a step of allodepletion.

A18. The composition of any preceding embodiment, wherein the genetically-modified T cells have an average vector copy number (VCN) of about 1 to 10 per cell.

A19. The composition of embodiment A18, wherein the genetically-modified T cells have an average VCN of about 1 to 7 per cell.

A20. The composition of embodiment A18, wherein the genetically-modified T cells have an average VCN of about 2 to 6 per cell.

A21. A composition comprising genetically modified CD3+ T cells, wherein the genetically modified CD3+ T cells comprise about 20% to about 40% CD4+ T cells and about 60% to about 80% CD8+ T cells, wherein
(i) the modified CD4+ T cells comprise
   (a) about 25% to about 45% naïve cells,
   (b) about 15% to about 30% T-central memory (CM) cells,
   (c) about 15% to about 30% T-effector memory (EM) cells,
   (d) about 2% to about 15% terminal effector memory (TEMRA) cells; and
(ii) the modified CD8+ T cells comprise
   (a) about 20% to about 60% naïve cells,
   (b) about 1% to about 10% CM cells,
   (c) about 1% to about 15% EM cells, and
   (d) about 10% to about 15% TEMRA cells.

A22. A composition comprising genetically modified CD3+ T cells, wherein
(i) about 20% to 40% of the T cells in the composition are CD8+ naïve cells;
(ii) about 1% to 20% of the T cells in the composition are CD8+ CM cells;
(iii) about 1% to 20% of the T cells in the composition are CD8+ EM cells; and
(iv) about 5% to 40% of the T cells in the composition are CD8+ TEMRA cells.

A23. The composition of embodiment A22 wherein about 20% to 30% of the T cells in the composition are CD8+ naïve cells.

A24. The composition of embodiments A22 or A23 wherein about 1% to 10% of the T cells in the composition are CD8+ CM cells.

A25. The composition of any of embodiments A22-A24 wherein about 1% to 10% of the T cells in the composition are CD8+ EM cells.

A26. The composition of any of embodiments A22-A25 wherein about 10% to 30% of the T cells in the composition are CD8+ TEMRA cells.

A27. A composition comprising genetically modified CD3+ T cells, wherein
(i) about 5% to 20% of the T cells in the composition are CD4+ naïve cells;
(ii) about 1% to 10% of the T cells in the composition are CD4+ CM cells;
(iii) about 1% to 10% of the T cells in the composition are CD4+ EM cells; and
(iv) about 1% to 5% of the T cells in the composition are CD4+ TEMRA cells.

A28. The composition of embodiment A27 wherein 5% to 15% of the T cells are CD4+ naïve cells.

A29. The composition of embodiment A27 or A28 wherein 1% to 7% of the T cells are CD4+CM cells.

A30. The composition of any of embodiments A27-A29 wherein 1% to 10% of the T cells are CD4+ EM cells.

A31. A method for treating a subject, comprising a step of introducing into the subject a composition of genetically-modified T cells of any preceding embodiment.

A32. A method for treating a subject, comprising a step of administering to the subject a pharmacological agent, wherein: (i) the subject has previously received an infusion of genetically-modified T cells according to any preceding embodiment; (ii) the pharmacological agent triggers the suicide switch; and (iii) the pharmacological agent is delivered at a dose which is high enough to kill at least 10% of genetically-modified T cells present in the subject, but low enough that at least 10% of genetically-modified T cells present in the subject survive. 10195) A33. A process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of terminal effector memory T cells, central memory T cells, and/or effector memory T cells relative to naïve T cells.

A34. The method of embodiment A33 wherein the method favours enrichment of terminal effector memory T cells relative to naïve T cells.

A35. A process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of CD8+ T cells relative to CD4+ T cells.

A36. A process for preparing genetically-modified T cells, comprising steps of: (i) culturing donor T cells in the presence of activating concentrations of IL 2, anti-CD3 antibody, and anti-CD28 antibody; (ii) after allowing a period of culture, adding further IL 2; (iii) after allowing a period of culture, introducing into the T cells DNA encoding both a suicide switch and a selectable marker; (iv) after allowing a period of culture, adding further IL 2; (v) selecting cells which express the selectable marker; (vi) after allowing a period of culture, adding further IL 2; (vii) harvesting the genetically-modified T cells; provided that steps (iv) and (vi) are optional.

A37. The process of any one of embodiments A33-A36, wherein the genetically-modified T cells have an average-VCN of about 1 to 10 per cell.

A38. The process of embodiment A37, wherein the genetically-modified T cells have an average VCN of about 1 to 7 per cell.

A39. The process of embodiment A36 wherein step (i) occurs at the start of the process (day 1).

A40. The process of embodiment A36 wherein step (iv) occurs on day 3 of the process.

A41. The process of embodiment A36 wherein step (vi) occurs on day 6 of the process.

A42. The process of and of embodiments A36-A41 wherein, at the end of the process, at least 50% of the cells are transduced and viable.

A43. The process of any of embodiments A36-A41 wherein, at the end of the process, at least 90% of the cells are transduced and viable.

It will be understood that the inventors' work has been described above by way of example only and modifications may be made while remaining within the scope and spirit of the invention.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

1. A composition comprising genetically-modified T cells comprising genetically-modified CD4+ T cells and genetically-modified CD8+ T cells, wherein:
(i) the genetically-modified T cells express a suicide switch; and
(ii) about 25% to 60% of the genetically-modified T cells are naïve T cells.

2. The composition of claim 1 wherein about 30-60% of the genetically-modified T cells are naïve T cells.

3. The composition of claim 1 wherein about 42-49% of the genetically-modified T cells are naïve T cells.

4. The composition of claim 1, wherein the ratio of genetically-modified CD4+ T cells to genetically-modified CD8+ T cells in the composition is less than 2.

5. The composition of any of the preceding claims, wherein at least 10% of the genetically-modified CD8+ T cells are terminal effector memory T cells and/or no more than 58% of the genetically-modified CD8+ T cells are naïve T cells.

6. The composition of claim 5, wherein at least 30% of the genetically-modified CD8+ T cells are terminal effector memory T cells and no more than 50% of the genetically-modified CD8+ T cells are naïve T cells.

7. The composition of any of the preceding claims, wherein the genetically-modified T cells display a range of expression levels of the cell surface transgene marker, wherein the range is at least 10-fold, wherein the expression levels are measured by flow cytometry, and wherein the expression levels are measured as mean fluorescence intensity (MFI) values.

8. The composition of claim 7, wherein the range is at least 100-fold.

9. The composition of claim 7 or 8, wherein the genetically-modified T cells display a range of sensitivities to a trigger molecule, such that exposure of the cells to a particular concentration of the trigger molecule leads to death of at least 10% of the cells but permits at least 10% of the cells to survive.

10. The composition of any preceding claim, wherein the ratio of genetically-modified CD4+ T cells to genetically-modified CD8+ T cells in the composition is less than 0.5.

11. The composition of any preceding claim, wherein the suicide switch comprises caspase-9.

12. The composition of any preceding claim, wherein the suicide switch comprises a FKBP12 region, a FKBP12 variant region, a FKBP12-Rapamycin Binding (FRB) or FRB variant region.

13. The composition of any one of claims 9-12, wherein the trigger molecule is rapamycin, a rapalog, AP1903, AP20187, or AP1510.

14. The composition of claim 12, wherein the FKBP12 variant region has an amino acid substitution at position 36 selected from the group consisting of valine, leucine, isoleuceine and alanine.

15. The composition of claim 13, wherein the FKBP12 variant region comprises two copies of FKBP12v36.

16. The composition of any preceding claim, wherein the genetically-modified T cells are human T cells.

17. The composition of any preceding claim, wherein: (i) the genetically-modified T cells express the iCasp9 suicide switch linked to the ΔCD19 marker, inserted into the genome of the T cells by retroviral transduction; (ii) the genetically-modified T cells have at least a 10-fold range of expression levels of a ΔCD19 cell surface marker measured by flow cytometry; (iii) the composition includes a greater number of CD8+ the genetically-modified T cells than CD4+ the genetically-modified T cells; (iv) the composition includes genetically-modified terminal effector memory T cells, genetically-modified T effector memory cells, and genetically-modified T central memory cells; (v) less than 50% of the genetically-modified T cells are naïve T cells; and (vi) the T cells were obtained from a human donor and were not subjected to a step of allodepletion.

18. The composition of any preceding claim, wherein the genetically-modified T cells have an average vector copy number (VCN) of about 1 to 10 per cell.

19. The composition of claim 18, wherein the genetically-modified T cells have an average VCN of about 1 to 7 per cell.

20. The composition of claim 18, wherein the genetically-modified T cells have an average VCN of about 2 to 6 per cell.

21. A composition comprising genetically modified CD3+ T cells, wherein the genetically modified CD3+ T cells comprise about 20% to about 40% CD4+ T cells and about 60% to about 80% CD8+ T cells, wherein
(i) the modified CD4+ T cells comprise
(a) about 25% to about 45% naïve cells,
(b) about 15% to about 30% T-central memory (CM) cells,
(c) about 15% to about 30% T-effector memory (EM) cells,
(d) about 2% to about 15% terminal effector memory (TEMRA) cells; and
(ii) the modified CD8+ T cells comprise
(a) about 20% to about 60% naïve cells,
(b) about 1% to about 10% CM cells,
(c) about 1% to about 15% EM cells, and
(d) about 10% to about 15% TEMRA cells.

22. A composition comprising genetically modified CD3+ T cells, wherein
(i) about 20% to 40% of the T cells in the composition are CD8+ naïve cells;
(ii) about 1% to 20% of the T cells in the composition are CD8+ CM cells;
(iii) about 1% to 20% of the T cells in the composition are CD8+ EM cells; and
(iv) about 5% to 40% of the T cells in the composition are CD8+ TEMRA cells.

23. The composition of claim 22 wherein about 20% to 30% of the T cells in the composition are CD8+ naïve cells.

24. The composition of claim 22 or 23 wherein about 1% to 10% of the T cells in the composition are CD8+ CM cells.

25. The composition of any of claims 22-24 wherein about 1% to 10% of the T cells in the composition are CD8+ EM cells.

26. The composition of any of claims 22-25 wherein about 10% to 30% of the T cells in the composition are CD8+ TEMRA cells.

27. A composition comprising genetically modified CD3+ T cells, wherein
(i) about 5% to 20% of the T cells in the composition are CD4+ naïve cells;
(ii) about 1% to 10% of the T cells in the composition are CD4+ CM cells;
(iii) about 1% to 10% of the T cells in the composition are CD4+ EM cells; and
(iv) about 1% to 5% of the T cells in the composition are CD4+ TEMRA cells.

28. The composition of claim 27 wherein 5% to 15% of the T cells are CD4+ naïve cells.

29. The composition of claim 27 or 28 wherein 1% to 7% of the T cells are CD4+ CM cells.

30. The composition of any of claims 27-29 wherein 1% to 10% of the T cells are CD4+ EM cells.

31. A method for treating a subject, comprising a step of introducing into the subject a composition of genetically-modified T cells of any preceding claim.

32. A method for treating a subject, comprising a step of administering to the subject a pharmacological agent, wherein:
(i) the subject has previously received an infusion of genetically-modified T cells according to any preceding claim;
(ii) the pharmacological agent triggers the suicide switch; and (iii) the pharmacological agent is delivered at a dose which is high enough to kill at least 10% of genetically-modified T cells present in the subject, but low enough that at least 10% of genetically-modified T cells present in the subject survive.

33. A process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of terminal effector memory T cells, central memory T cells, and/or effector memory T cells relative to naïve T cells.

34. The method of claim 33 wherein the method favours enrichment of terminal effector memory T cells relative to naïve T cells.

35. A process for preparing genetically-modified T cells, comprising steps of: (i) introducing nucleic acid into T cells from a donor subject, wherein the nucleic acid can direct expression of a suicide switch which can lead to cell death when the cells are exposed to a trigger molecule; and (ii) culturing the T cells under conditions which favour enrichment of CD8+ T cells relative to CD4+ T cells.

36. A process for preparing genetically-modified T cells, comprising steps of: (i) culturing donor T cells in the presence of activating concentrations of IL 2, anti-CD3 antibody, and anti-CD28 antibody; (ii) after allowing a period of culture, adding further IL 2; (iii) after allowing a period of culture, introducing into the T cells DNA encoding both a suicide switch and a selectable marker; (iv) after allowing a period of culture, adding further IL 2; (v) selecting cells which express the selectable marker; (vi) after allowing a period of culture, adding further IL 2; (vii) harvesting the genetically-modified T cells; provided that steps (iv) and (vi) are optional.

37. The process of any one of claims 33-36, wherein the genetically-modified T cells have an average VCN of about 1 to 10 per cell.

38. The process of claim 37, wherein the genetically-modified T cells have an average VCN of about 1 to 7 per cell.

39. The process of claim 36 wherein step (i) occurs at the start of the process (day 1).

40. The process of claim 36 wherein step (iv) occurs on day 3 of the process.

41. The process of claim 36 wherein step (vi) occurs on day 6 of the process.

42. The process of and of claims 36-41 wherein, at the end of the process, at least 50% of the cells are transduced and viable.

43. The process of any of claims 36-41 wherein, at the end of the process, at least 90% of the cells are transduced and viable.
